(19) United States
(12) Patent Application Publication
(10) Pub. No.: US 2025/0104877 A1
(43) Pub. Date: Mar. 27, 2025

(54) SYSTEM AND METHODS FOR THE DETERMINATION OF EFFECTIVE NUTRITIONAL SUPPLEMENTS TO IMPROVE PERFORMANCE AND WELL-BEING

(71) Applicants: Theodore WILEY, London (GB); Nicholas WILEY, Berkshire (GB)

(72) Inventors: Theodore WILEY, London (GB); Nicholas WILEY, Berkshire (GB)

(21) Appl. No.: 18/722,708

(22) PCT Filed: Dec. 21, 2022
(86) PCT No.: PCT/IB2022/062643

§ 371 Date: Jun. 21, 2024

Related U.S. Application Data
(60) Provisional application No. 63/292,777, filed on Dec. 22, 2021.

(51) Int. Cl. G16H 50/50; G16H 10/60; G16H 20/60

(52) U.S. Cl. CPC G16H 50/50; G16H 10/60; G16H 20/60

(57) ABSTRACT

Systems and methods for implementing a nutritional supplement plan for a user. The systems and methods involve analyzing qualitative and/or quantitative data of the user, such as genomic, blood serum, physiological, and/or well-being data, and utilizing a nutritional supplement model for achieving optimum levels of blood serum markers for a given supplement formulation, wherein the nutritional supplement model is iteratively updated based on actual levels of blood serum markers and updated genomic, blood serum, physiological, and/or well-being data the user after the user ingests the nutritional supplement. Such systems and methods can also be used to identify effective nutritional supplements for a user and achieve improvements in performance and/or health of the user.

### CROSS-REFERENCE TO RELATED APPLICATIONS

The present application claims the priority benefit of U.S. Provisional Patent Application Ser. No. 63/292,777, filed Dec. 22, 2021, entitled SYSTEM AND METHODS FOR THE DETERMINATION OF EFFECTIVE NUTRITIONAL SUPPLEMENTS TO IMPROVE PERFORMANCE AND WELL BEING, incorporated by reference in its entirety herein.

### BACKGROUND OF THE DISCLOSURE

### Field

The present disclosure relates to systems and methods for determination of effective nutritional supplements to improve performance and well-being of users. In particular, the present disclosure relates to systems and methods for implementing a personalized nutritional supplement plan for each user to improve performance and well-being of the user

### Description of Related Art

Research and analysis have identified links between nutrition and sports performance. Nutritional supplements are often necessary for athletes to meet their full potential. An International Olympic Committee (IOC) consensus statement published in British Journal of Sports Medicine in 2018 concluded that “Some supplements, when used appropriately may help athletes to meet sports nutrition goals, train hard, and stay healthy and injury-free. A few supplements can directly enhance competition performance. However, it takes considerable effort and expert knowledge to identify which products are appropriate, how to integrate them into the athlete's sports nutrition plan, and how to ensure that any benefits outweigh the possible negative side effects, including the potential for an ADRV (anti-doping rule violation).”

Nutrigenomics is the study of the effects of food and food constituents on gene expression, and how genetic variations affect the nutritional environment. It focuses on understanding the interactions between nutrients and other dietary bioactives with the genome at the molecular level, to understand how specific nutrients or dietary regimes may affect human health.

### BRIEF SUMMARY OF THE DISCLOSURE

The present disclosure relates to systems and methods for determination of effective nutritional supplements to improve performance and well-being of users. In particular, the present disclosure relates to systems and methods for implementing a personalized nutritional supplement plan for each user to improve performance and well-being of the user.

Systems and methods are provided for implementing a nutritional supplement plan for a user. In an example embodiment, a system is described that includes a non-transitory memory configured to store one or more of qualitative data or quantitative data including genomic data, blood serum data, physiological data, and well-being data of the user. The system further includes a processor. In some embodiments, the processor is configured to analyze the qualitative data or quantitative data including genomic data, blood serum data, physiological data, and well-being data of the user. In some embodiments, the processor is configured to utilize a nutritional supplement model for achieving optimized performance or well-being of the user, the nutritional supplement model including formulation for a nutritional supplement to be ingested by the user, the nutritional supplement including a plurality of ingredients. In some embodiments, the processor is configured to iteratively update the nutritional supplement model based on actual levels of one or more of blood serum markers, genomic data, blood serum data, physiological data, and well-being data of the user after the user ingests the nutritional supplement.

In some embodiments, the processor is further configured to train the nutritional supplement model via an artificial intelligence model to improve correlations in the nutritional supplement model.

In some embodiments, the artificial intelligence model is a machine learning model incorporating data sets of a population.

In some embodiments, the artificial intelligence model is selected from the group consisting of a linear regression model, a logistic regression model, a polynomial regression model, a linear discriminant analysis model, a decision tree model, a naïve bayes model, a K-nearest neighbor model, a learning vector quantization model, a support vector machine, a bagging and random forest model, and a deep neural network.

In some embodiments, the processor is further configured to implement gamification mechanisms to gather user performance data.

In some embodiments, the gamification mechanisms incorporate social engineering with the gamification mechanisms to incentivize the user to utilize the gamification mechanisms.

In some embodiments, the processor is further configured to attach adjustable weighting factors to ingredient terms in the nutritional supplement model based on nutritional supplement ingredient absorption rates of the user.

In some embodiments, the processor is further configured to impose upper limits and lower limits on amounts of the ingredients included in the nutritional supplement model.

In some embodiments, the system further includes a genetic material sampler configured to generate genomic data based on genetic material of the user.

In some embodiments, the system further includes a blood serum analyzer configured to generate the blood serum data of the user.

In another example embodiment, a method for implementing a nutritional supplement plan for a user is described. The method includes receiving one or more of qualitative data or quantitative data including genomic data, blood serum data, physiological data, and well-being data of the user. In some embodiments, the method includes analyzing the qualitative data or quantitative data including genomic data, blood serum data, physiological data, and well-being data of the user. In some embodiments, the method further includes utilizing a nutritional supplement model for achieving optimized performance or well-being of the user, the nutritional supplement model including formulation for a nutritional supplement to be ingested by the user, the nutritional supplement including a plurality of ingredients. In some embodiments, the method includes iteratively updating the nutritional supplement model based on actual levels of one or more of blood serum markers, genomic data, blood serum data, physiological data, and well-being data of the user after the user ingests the nutritional supplement.

In yet another example embodiment, a system for implementing a nutritional supplement plan for a user is described. The system includes a genetic material sampler configured to generate genomic data based on genetic material of the user and a blood serum analyzer configured to generate blood serum data of the user. The system further includes a non-transitory memory configured to store qualitative data including the genomic data, the blood serum data, physiological data, and well-being data of the user. The system also includes a processor. The processor is configured to analyze the qualitative data including genomic data, blood serum data, physiological data, and well-being data of the user. In some embodiments, the processor is further configured to construct a nutritional supplement model for achieving optimized performance or well-being of the user, the nutritional supplement model including formulation for a nutritional supplement to be ingested by the user, the nutritional supplement including a plurality of ingredients. In some embodiments, the processor is configured to iteratively update the nutritional supplement model based on one or more of actual levels of blood serum markers, genomic data, blood serum data, physiological data, and well-being data of the user after the user ingests the nutritional supplement. In some embodiments, the processor is configured to attach adjustable weighting factors to ingredient terms in the nutritional supplement model based on nutritional supplement ingredient absorption rates of the user. In some embodiments, the processor is configured to impose upper limits and lower limits on amounts of the ingredients included in the nutritional supplement model. In some embodiments, the processor is configured to train the nutritional supplement model via an artificial intelligence model to improve correlations in the nutritional supplement model. In some embodiments, the processor is configured to implement gamification mechanisms to gather user performance data. In some embodiments, the processor is configured to incorporate social engineering with the gamification mechanisms to incentivize the user to utilize the gamification mechanisms.

Other aspects and advantages of the disclosure will become apparent from the following detailed description, taken in conjunction with the accompanying drawings, which illustrate by way of example, the principles of the disclosure.

### BRIEF DESCRIPTION OF THE DRAWINGS

The foregoing and other objects, aspects, features, and advantages of the disclosure will become more apparent and better understood by referring to the following description taken in conjunction with the accompanying drawings, in which:

FIG. 1A is a block diagram depicting an embodiment of a network environment comprising client device in communication with server device;

FIG. 1B is a block diagram depicting a could computing environment comprising client device in communication with cloud service providers;

FIG. 1C and FIG. 1D are block diagrams depicting embodiments of computing devices useful in connection with the methods and systems described herein;

FIG. 2A depicts an implementation of some of a server and client architecture of a system for implementing a nutritional supplement plan for a user, according to one or more embodiments;

FIG. 2B depicts a block flow diagram for determination of formulation for a nutritional supplement, according to one or more embodiments;

FIG. 3 depicts an exemplary nutritional supplement model, according to one or more embodiments;

FIG. 4 illustrates an example of formulation for a nutritional supplement to be ingested by a user, according to one or more embodiments;

FIG. 5 illustrates a machine learning model, according to one or more embodiments;

FIG. 6 depicts a flow diagram for creating a nutritional supplement for a user, according to one or more embodiments;

FIG. 7 depicts an example of a user interface that a user may use to view his or her health report, according to one or more embodiments;

FIG. 8 depicts an example of a user interface that a user may use to view personalized formulation of a nutritional supplement, according to one or more embodiments;

FIG. 9 depicts an example of a user interface that a user may use to view his or her nutrition report, according to one or more embodiments;

FIG. 10 depicts an example of a user interface showing vitamin B6 requirement for a user, according to one or more embodiments;

FIG. 11 depicts a flowchart for iteratively updating a nutritional supplement model for a user after the user ingests a nutritional supplement, according to one or more embodiments; and

FIG. 12A and FIG. 12B depict a flowchart for training a nutritional supplement model via an artificial intelligence model to improve correlations in the nutritional supplement model, according to some embodiments.

### DETAILED DESCRIPTION

For purposes of reading the description of the various embodiments below, the following descriptions of the sections of the specifications and their respective contents may be helpful:

Section A describes a network environment and computing environment which may be useful for practicing embodiments described herein.

Section B describes embodiments of systems and methods for determination of effective nutritional supplements to improve performance and well-being of users. In particular, Section B describes systems and methods for implementing a personalized nutritional supplement plan for each user to improve performance and well-being of the user.

### A. Computer and Network Environment

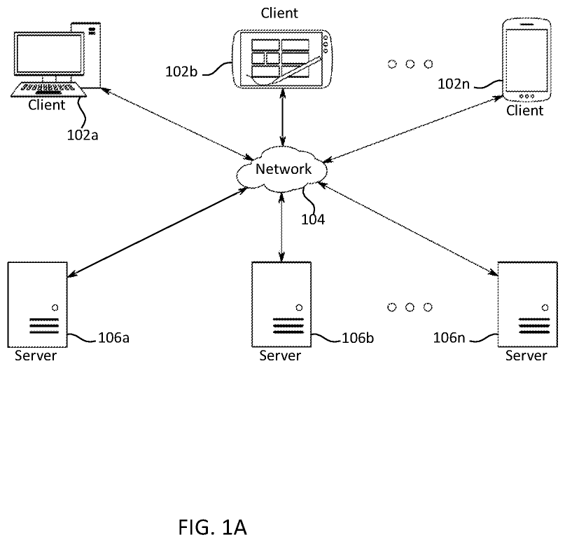

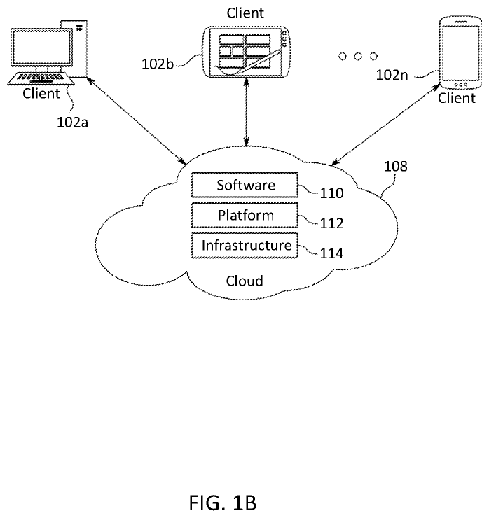

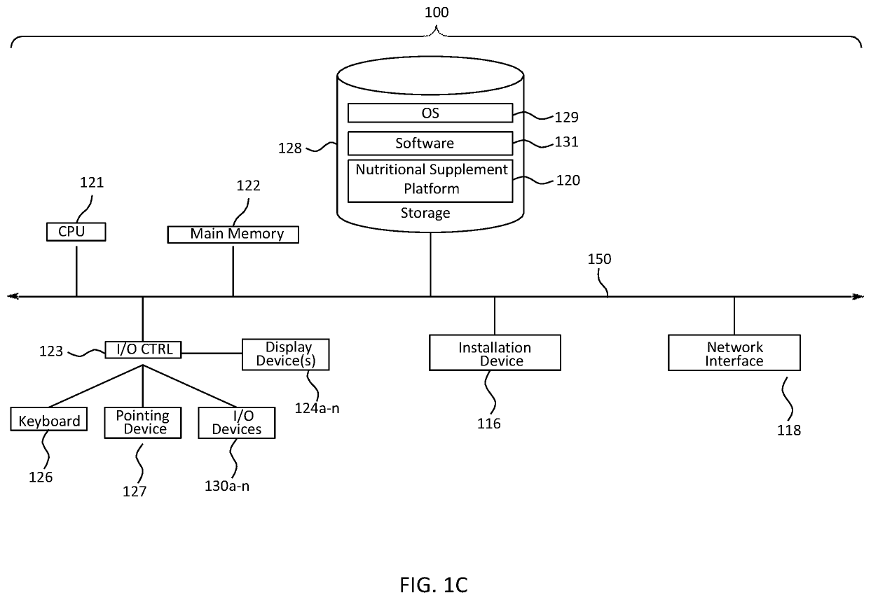

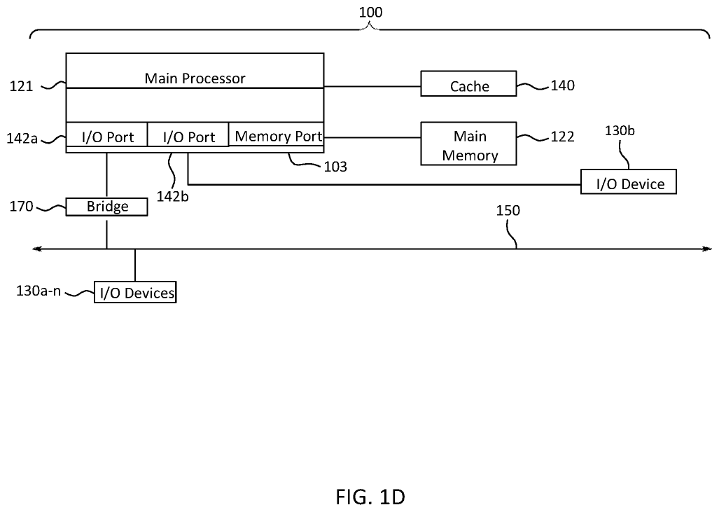

Prior to discussing specific embodiments of the present solution, it may be helpful to describe aspects of the operating environment as well as associated system components (e.g., hardware elements) in connection with the methods and systems described herein. Referring to FIG. 1A, an embodiment of a network environment is depicted. In a brief overview, the network environment includes one or more clients 102a-102n (also generally referred to as local machines(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node(s) 106, machine(s) 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 1A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ may be a public network. In still another of these embodiments, networks 104 and 104′ may both be private networks.

The network 104 may be connected via wired or wireless links. Wired links may include Digital Subscriber Line (DSL), coaxial cable lines, or optical fiber lines. Wireless links may include Bluetooth®, Bluetooth Low Energy (BLE), ANT/ANT+, ZigBee, Z-Wave, Thread, Wi-Fi®, Worldwide Interoperability for Microwave Access (WiMAX®), mobile WiMAX®, WiMAX®-Advanced, NFC, SigFox, LoRa, Random Phase Multiple Access (RPMA), Weightless-N/P/W, an infrared channel, or a satellite band. The wireless links may also include any cellular network standards to communicate among mobile devices, including standards that qualify as 1G, 2G, 3G, 4G, or 5G. The network standards may qualify as one or more generations of mobile telecommunication standards by fulfilling a specification or standards such as the specifications maintained by the International Telecommunication Union. The 3G standards, for example, may correspond to the International Mobile Telecommuniations-2000 (IMT-2000) specification, and the 4G standards may correspond to the International Mobile Telecommunication Advanced (IMT-Advanced) specification. Examples of cellular network standards include AMPS, GSM, GPRS, UMTS, CDMA2000, CDMA-1×RTT, CDMA-EVDO, LTE, LTE-Advanced, LTE-M1, and Narrowband IoT (NB-IoT). Wireless standards may use various channel access methods, e.g., FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network may vary widely and the network 104 can be a body area network (BAN), a personal area network (PAN), a local-area network (LAN), e.g., Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv4 and IPV6), or the link layer. The network 104 may be a type of broadcast network, a telecommunications network, a data communication network, or a computer network.

In some embodiments, the system may include multiple, logically grouped servers 106. In one of these embodiments, the logical group of servers may be referred to as a server farm or a machine farm. In another of these embodiments, the servers 106 may be geographically dispersed. In other embodiments, a machine farm may be administered as a single entity. In still other embodiments, the machine farm includes a plurality of machine farms. The servers 106 within each machine farm can be heterogeneous-one or more of the servers 106 or machines 106 can operate according to one type of operating system platform (e.g., Windows, manufactured by Microsoft Corp. of Redmond, Washington), while one or more of the other servers 106 can operate according to another type of operating system platform (e.g., Unix, Linux, or Mac OSX).

In one embodiment, servers 106 in the machine farm may be stored in high-density rack systems, along with associated storage systems, and located in an enterprise data center. In this embodiment, consolidating the servers 106 in this way may improve system manageability, data security, the physical security of the system, and system performance by locating servers 106 and high-performance storage systems on localized high-performance networks. Centralizing the servers 106 and storage systems and coupling them with advanced system management tools allows more efficient use of server resources.

The servers 106 of each machine farm do not need to be physically proximate to another server 106 in the same machine farm. Thus, the group of servers 106 logically grouped as a machine farm may be interconnected using a wide-area network (WAN) connection or a metropolitan-area network (MAN) connection. For example, a machine farm may include servers 106 physically located in different continents or different regions of a continent, country, state, city, campus, or room. Data transmission speeds between servers 106 in the machine farm can be increased if the servers 106 are connected using a local-area network (LAN) connection or some form of direct connection. Additionally, a heterogeneous machine farm may include one or more servers 106 operating according to a type of operating system, while one or more other servers execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alta, California; the Xen hypervisor, an open source product whose development is overseen by Citrix Systems, Inc. of Fort Lauderdale, Florida; the HYPER-V hypervisors provided by Microsoft, or others. Hosted hypervisors may run within an operating system on a second software level. Examples of hosted hypervisors may include VMWare Workstation and VirtualBox, manufactured by Oracle Corporation of Redwood City, California.

Management of the machine farm may be de-centralized. For example, one or more servers 106 may comprise components, subsystems, and modules to support one or more management services for the machine farm. In one of these embodiments, one or more servers 106 provide functionality for management of dynamic data, including techniques for handling failover, data replication, and increasing the robustness of the machine farm. Each server 106 may communicate with a persistent store and, in some embodiments, with a dynamic store. Server 106 may be a file server, application server, web server, proxy server, appliance, network appliance, gateway, gateway server, virtualization server, deployment server, SSL VPN server, or firewall. In one embodiment, a plurality of servers 106 may be in the path between any two communicating servers 106.

Referring to FIG. 1B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or zero client may depend on the connection to the cloud 108 or server 106 to provide functionality. A zero client may depend on the cloud 108 or other networks 104 or servers 106 to retrieve operating system data for the client device 102. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 109 may include both the private and public networks 104 and servers 106.

The cloud 108 may also include a cloud-based delivery, e.g., Software as a Service (SaaS) 110, Platform as a Service (PaaS) 112, and Infrastructure as a Service (IaaS) 114. IaaS may refer to a user renting the user of infrastructure resources that are needed during a specified time period. IaaS may offer storage, networking, servers, or virtualization resources from large pools, allowing the users to quickly scale up by accessing more resources as needed. Examples of IaaS include Amazon Web Services (AWS) provided by Amazon, Inc. of Seattle, Washington, Rackspace Cloud provided by Rackspace Inc. of San Antonio, Texas, Google Compute Engine provided by Google Inc. of Mountain View, California, or RightScale provided by RightScale, Inc. of Santa Barbara, California. PaaS providers may offer functionality provided by IaaS, including, e.g., storage, networking, servers, or virtualization, as well as additional resources, e.g., the operating system, middleware, or runtime resources. Examples of PaaS include Windows Azure provided by Microsoft Corporation of Redmond, Washington, Google App Engine provided by Google Inc., and Heroku provided by Heroku, Inc. of San Francisco California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include Google Apps provided by Google Inc., Salesforce provided by Salesforce.com Inc. of San Francisco, California, or Office365 provided by Microsoft Corporation. Examples of SaaS may also include storage providers, e.g., Dropbox provided by Dropbox Inc. of San Francisco, California, Microsoft OneDrive provided by Microsoft Corporation, Google Drive provided by Google Inc., or Apple iCloud provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g., Google Chrome, Microsoft Internet Explorer, or Mozilla Firefox provided by Mozilla Foundation of Mountain View, California). Clients 102 may also access SaaS resources through smartphone or tablet applications, including e.g., Salesforce Sales Cloud, or Google Drive App. Clients 102 may also access SaaS resources through the client operating system, including e.g., Windows file system for Dropbox.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g., a computer, network device or appliance capable of communicating on any type and form of network and performing the operations described herein.

FIG. 1C and FIG. 1D depict block diagrams of a computing device 100 useful for practicing an embodiment of the client 102 or a server 106. As shown in FIG. 1C and FIG. 1D, each computing device 100 includes a central processing unit 121, and a main memory unit 122. As shown in FIG. 1C, a computing device 100 may include a storage device 128, an installation device 116, a network interface 118, and I/O controller 123, display devices 124a-124n, a keyboard 126 and a pointing device 127, e.g., a mouse. The storage device 128 may include, without limitation, an operating system 129, software 131, and software of a nutritional supplement platform 120. As shown in FIG. 1D, each computing device 100 may also include additional optional elements, e.g., a memory port 103, a bridge 170, one or more input/output devices 130a-130n (generally referred to using reference numeral 130), and a cache memory 140 in communication with the central processing unit 121.

The central processing unit 121 is any logic circuitry that responds to, and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit 122 may include one or more memory chips capable of storing data and allowing any storage location to be directly accessed by the microprocessor 121. Main memory unit 122 may be volatile and faster than storage 128 memory. Main memory units 122 may be Dynamic Random-Access Memory (DRAM) or any variants, including Static Random-Access Memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile Random Access Memory (NVRAM), flash memory non-volatile static RAM (nvSRAM), Ferroelectric RAM (FeRAM), Magnetoresistive RAM (MRAM), Phase-change RAM (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 1C, the processor 121 communicates with main memory 122 via a system bus 150 (described in more detail below). FIG. 1D depicts an embodiment of a computing device 100 in which the processor communicates directly with main memory 122 via a memory port 103. For example, in FIG. 1D the main memory 122 may be DRDRAM.

FIG. 1D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 1D, the processor 121 communicates with various I/O devices 130 via a local system bus 150. Various buses may be used to connect the central processing unit 121 to any of the I/O devices 130, including a PCI bus, a PCI-X bus, a PCI-Express bus, or a NuBus. For embodiments in which the I/O device is a video display 124, the processor 121 may use an Advanced Graphic Port (AGP) to communicate with the display 124 or the I/O controller 123 for the display 124. FIG. 1D depicts an embodiment of a computer 100 in which the main processor 121 communicates directly with I/O device 130b or other processors 121′ via HYPERTRANSPORT, RAPIDIO, or INFINIBAND communications technology. FIG. 1D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex cameras (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple iPhone. Some devices 130a-130n allow gesture recognition inputs through combining some of the inputs and outputs. Some devices 130a-130n provide for facial recognition which may be utilized as an input for different purposes including authentication and other commands. Some devices 130a-130n provide for voice recognition and inputs, including, e.g., Microsoft KINECT, SIRI for iPhone by Apple, Google Now or Google Voice Search, and Alexa by Amazon.

Additional devices 130a-130n have both input and output capabilities, including, e.g., haptic feedback devices, touchscreen displays, or multi-touch displays. Touchscreen displays, multi-touch displays, touchpads, touch mice, or other touch sensing devices may use different technologies to sense touch, including, e.g., capacitive, surface capacitive, projected capacitive touch (PCT), in cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augmented reality devices. An I/O controller may control the I/O devices 123 as shown in FIG. 1C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, a I/O device 130 may be a bridge between the system bus 150 and an external communication bus, e.g., a USB bus, a SCSI bus, a FireWire bus, an Ethernet bus, a Gigabit Ethernet bus, a Fiber Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode (LED) displays, digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g., stereoscopy, polarization filters, active shutters, or auto stereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect, or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments, software may be designed and constructed to use another computer's display device as a second display device 124a for the computing device 100. For example, in one embodiment, an Apple iPad may connect to a computing device 100 and use the display of the device 100 as an additional display screen that may be used as an extended desktop. One OF ordinarily skill in the art will recognize and appreciate the various ways and embodiments that a computing device 100 may be configured to have multiple display devices 124a-124n.

Referring again to FIG. 1C, the computing device 100 may comprise a storage device 128 (e.g., one or more hard disk drives or redundant arrays of independent disks) for storing an operating system or other related software, and for storing application software programs such as any program related to the software of a nutritional supplement platform 120. Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage devices 128 may be non-volatile, mutable, or read-only. Some storage devices 128 may be internal and connect to the computing device 100 via a bus 150. Some storage devices 128 may be external and connect to the computing device 100 via a I/O device 130 that provides an external bus. Some storage devices 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage devices 128 may also be used as an installation device 116 and may be suitable for installing software and programs. Additionally, the operating system and the software can be run from a bootable medium, for example, a bootable CD, e.g., KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, InfiniBand), broadband connections (e.g., ISDN, Frame Relay, ATM, Gigabit Ethernet, Ethernet over SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMAX, and direct asynchronous connections). In one embodiment, the computing device 100 communicates with other computing devices 100′ via any type and/or form of gateway or tunneling protocol e.g., Secure Socket Layer (SSL) or Transport Layer Security (TLS), or the Citrix Gateway Protocol manufactured by Citrix Systems, Inc. The network interface 118 may comprise a built-in network adapter, network interface card, PCMCIA network card, EXPRESSCARD network card, card bus network adapter, wireless network adapter, USB network adapter, modem, or any other device suitable for interfacing the computing device 100 to any type of network capable of communication and performing the operations described herein.

A computing device 100 of the sort depicted in FIG. 1B and FIG. 1C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2012, WINDOWS CE, WINDOWS Phone, WINDOWS XP, WINDOWS VISTA, and WINDOWS 7, WINDOWS RT, WINDOWS 8 and WINDOW 10, all of which are manufactured by Microsoft Corporation of Redmond, Washington; MAC OS and iOS, manufactured by Apple, Inc.; and Linux, a freely-available operating system, e.g., Linux Mint distribution (“distro”) or Ubuntu, distributed by Canonical Ltd. of London, United Kingdom; or Unix or other Unix-like derivative operating systems; and Android, designed by Google Inc., among others. Some operating systems, including, e.g., the CHROME OS by Google Inc., may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gamification system, mobile computing device, or any other type and/or form of computing, telecommunications or media device that is capable of communication. The computer system 100 has sufficient processor power and memory capacity to perform the operations described herein. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gamification system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, or a NINTENDO DS, NINTENDO 3DS, NINTENDO WII, or a NINTENDO WII U device manufactured by Nintendo Co., Ltd., of Kyoto, Japan, or an XBOX 360 device manufactured by Microsoft Corporation.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gamification system or any functionality made available by an application from a digital application distribution platform. For example, the iPod Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple lossless audio file formats and .mov, .m4v, and .mp4 MPEG-4 (H.264/MPEG-4 AVC) video file formats.

In some embodiments, the computing device 100 is a tablet e.g., the IPAD line of devices by Apple; GALAXY TAB family of devices by Samsung; or KINDLE FIRE, byAmazon.com, Inc. of Seattle, Washington. In other embodiments, the computing device 100 is an eBook reader, e.g., the KINDLE family of devices by Amazon.com, or NOOK family of devices by Barnes & Noble, Inc. of New York City, New York.

In some embodiments, the communications device 102 includes a combination of devices, e.g., a smartphone combined with a digital audio player or portable media player. For example, one of these embodiments is a smartphone, e.g., the iPhone family of smartphones manufactured by Apple, Inc.; a Samsung GALAXY family of smartphones manufactured by Samsung, Inc; or a Motorola DROID family of smartphones. In yet another embodiment, the communications device 102 is a laptop or desktop computer equipped with a web browser and a microphone and speaker system, e.g., a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in network 104 is monitored, as part of network management. In one of these embodiments, the status of a machine may include an identification of load information (e.g., the number of processes on the machine, CPU, and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

### B. Systems and Methods for Determination of Effective Nutritional Supplements to Improve Performance and Well-being

The present disclosure relates to systems and methods for determination of effective nutritional supplements to improve performance and well-being of users. In particular, the present disclosure relates to systems and methods for implementing a personalized nutritional supplement plan for each user to improve the performance and well-being of the user.

Research and analysis have identified links between nutrition and sports performance. Typically, nutritional supplements are necessary for athletes to meet their full potential. A nutritional supplement is a product that supplements a normal diet and is a source of macronutrients and/or micronutrients. A nutritional supplement may be used to correct a nutritional deficiency, to maintain an adequate intake of certain nutrients, or to support specific physiological functions. A nutritional supplement is often taken in oral form and is formulated and delivered as a dose.

In examples, nutritional supplements, when used appropriately, may help athletes to meet sports nutrition goals, to train hard, and to stay healthy and injury-free. Additionally, nutritional supplements may directly enhance competition performance. However, it takes considerable effort and expert knowledge to identify which nutritional supplements are appropriate, how to integrate the nutritional supplements into a nutritional plan of an athlete, and how to ensure that any benefits outweigh the possible negative side effects, including the potential for an anti-doping rule violation (ADRV). Moreover, the existing market for nutritional supplements is dominated by generic products that are “one size fits all” (OSFA) and are not tailored to a user's needs or objectives, and do not cater to the physiological factors and supplementation needs that differ from user to user. Consequently, users end up purchasing and taking nutritional supplements that may not have a positive impact on performance, health, and general well-being to the extent expected and may actually be detrimental in some cases. Moreover, such OSFA products are often not formulated with respect to clinically efficacious dosage of such nutritional supplements (i.e., the minimum quantity of a compound that has shown relative benefit to the consumer in the scientific literature) and are neither formulated nor continuously reformulated based on the impact or potential impact the products have on individuals in terms of their biological markers, performance, and overall well-being. This further reinforces the sub-optimal benefits of such OSFA products and may additionally lead to individuals abandoning a product they find ineffective or one which leads to adverse reactions.

Notably, a key problem with OSFA nutritional supplements is that they can be easily replicated by other market individuals if their ingredients and associated dosages are known. Suppliers of such nutritional supplements often rely upon “proprietary formulae” labeling to avoid having to disclose a full list of ingredients or indeed the actual quantities of each ingredient contained in the nutritional supplement. This lack of transparency can lead to individuals experiencing adverse reactions that may imperil their health and well-being or have adverse consequences, such as an athlete failing doping control tests for banned substances. Furthermore, recent data have indicated that one in five nutritional supplements is tainted with banned substances.

Nutrigenomics is the study of the effects of food and food constituents on gene expression, and how genetic variations affect the nutritional environment. Nutrigenomics focuses on understanding the interaction between nutrients and other dietary bioactives with the genome at the molecular level, to understand how specific nutrients or dietary regimes may affect human health. Examples of such effects can be seen when considering compounds such as vitamin B6, vitamin D, and iron. By controlling the intake of vitamin B6, vitamin D, and iron, it may be possible to optimize performance by reducing the likelihood of deficiency. In examples, the following example of optimizing performance (or reducing deficiencies) of a user by reducing the likelihood of deficiency may be considered.

TABLE 1
A. Vitamin B6: Between 58% and 73% of athletes are deficient in vitamin B6.
Outcome: Deficiency can lead to a significant decrease in VO₂ max and the anaerobic
threshold in less than four weeks.
Genotype Effect: An enzyme in methyl cycle, Methylene tetrahydrofolate reductase
(MTHFR) is involved in folate metabolism. Differing versions of this gene create
different enzyme efficiency, which may increase vitamin B needs. Homozygous
rs1801133(T; T) individuals have ~30% of the expected MTHFR enzyme activity, and
rs1801133(C; T) heterozygotes have ~65% activity, compared to the most common
genotype, rs1801133(C; C). (The rs1801133 is a single nucleotide polymorphism that
encodes a variant in MTHFR gene)
B. Vitamin D: Only ~5% of college athletes meet the RDA for vitamin D from food alone and
41% of the US general population are clinically deficient.
Outcome: Deficiency impairs muscle strength and causes type II muscle fiber atrophy
treatment increased endurance performance by 17.4% and 100 m performance by 7.4%.
Genotype Effect: The NADSYN1 gene encodes nicotinamide adenine dinucleotide
synthetase 1 (NADSYN1), which is one of the glutamine-dependent enzymes involved
in cholesterol synthesis and favors the production of nicotinamide adenine dinucleotide
(NAD+), the main coenzyme required for energy production and lipid synthesis. A
recent study revealed that variants near genes involved in cholesterol synthesis influence
the vitamin D status. The rs12785878 GG genotype was associated with an increased
risk of vitamin D insufficiency and of dyslipidemia.
C. Iron: Up to 50% of female athletes and a third of male athletes experience iron deficiency.
Outcome: Iron deficiency alone can reduce aerobic performance by an average of 4%.
Genotype Effect: The HFE gene regulates the liver protein hepcidin, which is
considered to be the principal iron-regulatory hormone. Levels of hepcidin increase
when iron levels increase in the blood, preventing intestinal cells and macrophages from
releasing iron into the bloodstream. When iron builds up in the cells, signals are
produced to reduce the iron absorption from food. Hepcidin levels decrease when the
body needs more iron. Mutations in the HFE gene such as C282Y and H63D decrease
hepcidin levels, causing intestinal cells to think more iron is needed. Thus, more iron is
absorbed. (rs1799945, rs1800562).

The above example in Table 1 demonstrates the effect of only three compounds (i.e., vitamin B6, vitamin D, and iron), however, the example indicates that by controlling nutritive variables through the provision of a personalized nutritional supplement plan, the performance of the user may be optimized as the likelihood of deficiency is reduced.

Accordingly, there is a need for systems and methods that can accurately formulate nutritional supplements tailored to cater to the physiological needs and performance goals of users, and to reformulate these nutritional supplements on a periodic basis in light of the knowledge of their impact obtained through feedback cycles envisaged on performance and well-being of the users.

The present disclosure describes systems and methods for determination of effective nutritional supplements to improve performance and well-being of users. The users may be understood as consumers of the nutritional supplements. According to an aspect, the present disclosure describes systems that include a multi-channel data collection and analytical platform incorporating an artificial intelligence model. The systems create personalized formulations of nutritional supplements for each user to improve performance and well-being of the user.

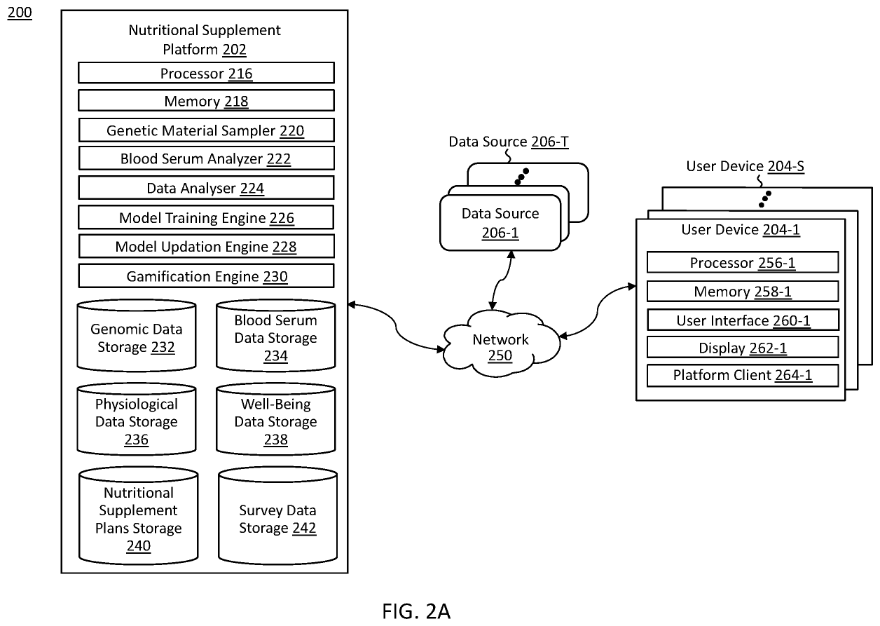

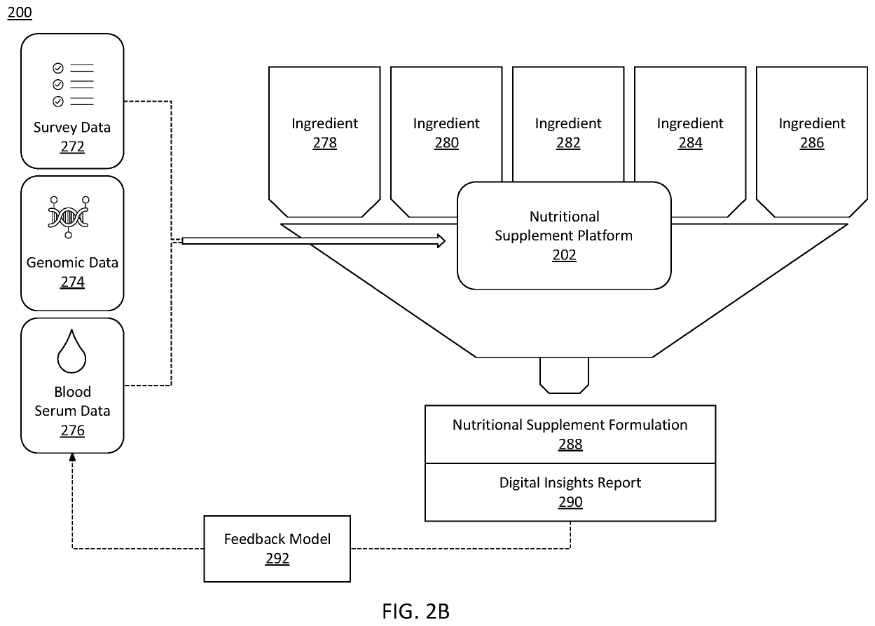

Referring to FIG. 2A, in a general overview, FIG. 2A depicts some of the server and client architecture of an implementation of system 200 for implementing a nutritional supplement plan for a user, according to one or more embodiments. System 200 may include nutritional supplement platform 202, user device(s) 204-(1-S), data sources 206-(1-T), and network 250 enabling communication between the system components for information exchange. Network 250 may be an example or instance of network 104, details of which are provided with reference to FIG. 1A and its accompanying description.

According to some embodiments, nutritional supplement platform 202 may be a multi-channel data collection and analytical platform that processes data from multiple sources and generates nutritional supplement plans for one or more users. In an example, a nutritional supplement plan includes information about normal dietary food and/or nutritional supplements to be consumed by a user to improve performance and well-being of the user. In examples, the nutritional supplement plan may define quantities (amount), types, and timings of nutritional supplements to be consumed. The nutritional supplement plan may be developed such that the user is enabled to achieve a nutritional goal while improving performance of the user. In examples, the user may be a consumer of the nutritional supplements. An example of the user may include an athlete. According to an implementation, a nutritional supplement plan for a user may be individually tailored, personalized, or customized.

In an implementation, nutritional supplement platform 202 may be implemented in a variety of computing systems, such as a mainframe computer, a server, a network server, a laptop computer, a desktop computer, a notebook, a workstation, and the like. In an implementation, nutritional supplement platform 202 may be implemented in a server, such as server 106 shown in FIG. 1A. In some implementations, nutritional supplement platform 202 may be implemented by a device, such as computing device 100 shown in FIG. 1C and FIG. 1D. In some embodiments, nutritional supplement platform 202 may be implemented as a part of a cluster of servers. In some embodiments, nutritional supplement platform 202 may be implemented across a plurality of servers, thereby, tasks performed by nutritional supplement platform 202 may be performed by the plurality of servers. These tasks may be allocated among the cluster of servers by an application, a service, a daemon, a routine, or other executable logic for task allocation. In some implementations, nutritional supplement platform 202 may be owned or managed or otherwise associated with an organization or any entity authorized thereof.

According to an embodiment, nutritional supplement platform 202 may include processor 216 and memory 218. For example, processor 216 and memory 218 of nutritional supplement platform 202 may be CPU 121 and main memory 122, respectively, as shown in FIG. 1C and FIG. 1D. Further, nutritional supplement platform 202 may include genetic material sampler 220, blood serum analyzer 222, data analyzer 224, model updation engine 228, model training engine 226, and gamification engine 230.

In some embodiments, genetic material sampler 220, blood serum analyzer 222, data analyzer 224, model training engine 226, model updation engine 228, and gamification engine 230 may be implemented in hardware, instructions executed by a processing module, or by a combination thereof. Processor 216 may comprise a computer, a processor, a state machine, a logic array, or any other suitable devices capable of processing instructions. Processor 216 may comprise a general-purpose processor which executes instructions to cause the general-purpose processor to perform the required tasks or, the processing module may be dedicated to performing the required functions. In some embodiments, genetic material sampler 220, blood serum analyzer 222, data analyzer 224, model training engine 226, model updation engine 228, and gamification engine 230 may comprise machine-readable instructions which, when executed by a processor/processing module, perform intended functionalities of genetic material sampler 220, blood serum analyzer 222, data analyzer 224, model training engine 226, model updation engine 228, and gamification engine 230. The machine-readable instructions may be stored on an electronic memory device, hard disk, optical disk, or other machine-readable storage medium or non-transitory medium. In an implementation, the machine-readable instructions may also be downloaded to the storage medium via a network connection, for example over network 250.

According to an implementation, to implement a nutritional supplement plan for a user, genetic material sampler 220 may be configured to generate genomic data based on genetic material of the user. In an implementation, genetic material sampler 220 may generate the genomic data based on salivary, cheek-swab, and blood-based methods of obtaining samples. In examples, a salivary testing kit may be provided to the user for collecting one or more salivary samples. In an implementation, genetic material sampler 220 may process one or more salivary samples of the user to identify single nucleotide polymorphisms (SNPs). An SNP is a type of polymorphism involving variation of a single base pair. A base pair consists of two complementary DNA nucleotide bases that pair together to form a “rung” of the DNA ladder. A nucleotide is the basic structural unit and building block for DNA. These building blocks are hooked together to form a chain of DNA. A nucleotide comprises three parts: a five-sided sugar, a phosphate group, and a nitrogenous (nitrogen containing) base. The sugar and phosphate group make up the backbone of the DNA double helix, while the bases are located in the middle. A chemical bond between the phosphate group of one nucleotide and the sugar of a neighboring nucleotide holds the backbone together. Chemical bonds (for example, hydrogen bonds) between the bases that are across from one another hold the two strands of the double helix together. In an implementation, genetic material sampler 220 may categorize the SNPs as either training SNPs or nutritional SNPs. The sample may be analyzed using various methodologies including next-generation sequencing (NGS), chromatin immunoprecipitation (ChIP) assays, targeted resequencing, and genotyping solutions. Outputs of the above methods will be fed into the computer algorithm and distinct units of the genome (SNPs) identifies as relevant, important, or associated with either nutritional status and training/physical performance will be separated. The system will assess and recognize the genotype of the user for each SNP and apply the appropriate score (as defined in the bespoke algorithm and scoring methodology). In examples, analysis of SNPs may be used to determine physiology of the user. For example, analysis of SNPs may be used to determine an ability of the user to absorb macronutrients and/or micronutrients, susceptibility of the user to certain stimuli, and possible disposition to injury. In examples, the stimuli may include physiological responses and rate of adaptation to exercise modalities, such as strength, endurance, power, and speed-based training. In an example implementation, genetic material sampler 220 may store the genomic data of the user in genomic data storage 232.

In an implementation, blood serum analyzer 222 may be configured to generate blood serum data of the user. According to an implementation, blood serum analyzer 222 may perform blood serum marker testing to generate blood serum data of the user. In an exemplary scenario, a finger prick blood testing kit may be provided to the user for collecting blood of the user required for blood serum marker testing. In examples, blood serum marker testing may be performed to determine actual levels of blood serum markers in the blood of the user at the time of testing. A blood serum marker is a marker that is present in blood serum as a result of the presence or absence of a compound (such as a micronutrient), a disease or ailment, or an object such as a tumor. In some cases, concentration of one or more blood serum markers may be used to calculate or infer a quantity of a compound which is present in the body of a user. In an example, one or more blood serum markers may indicate direct or indirect presence of a compound present in the body of a user. In an example implementation, blood serum analyzer 222 may store blood serum data of the user in blood serum data storage 234. According to an implementation, the process of generation of the genomic data and blood serum data may be performed in real-time (i.e., whenever a nutritional supplement plan is to be implemented for the user). In examples, a user may provide both a salivary sample and (in some instances) a blood serum sample. In examples, the salivary and/or blood serum sample may be provided to nutritional supplement platform 202 and may be utilized to develop one or multiple supplement products and/or a supplement plan. In examples, a user may provide feedback in the form of athletic performance data, subjective qualitative information, and (in some cases) additional blood serum samples for analysis following the initial outputs. In examples, this data may be utilized to make alterations to the original supplement product formulations, plans, and recommendations, for example by model training engine 226 to establish enhanced associative models. In examples, there is a “real-time” or immediate provision of a supplement product/plan and in other cases, non-“real-time” applications.

In some examples, biological samples may be stored in a biobank for future analysis depending on the initial methodology or technology used to analyze the material. For example, nutritional supplement platform 202 may opt to use a dedicated array to analyze a subset of SNPs instead of wide-scale whole genome sequencing (this could be due to cost implications of the technology). In examples samples may be stored and re-analyzed in future to establish a larger dataset from which to provide enhanced recommendations to customers when it becomes viable to do so. According to some implementations, the process of generation of genomic data and blood serum data may not be performed in real-time. In examples, a user may opt to purchase additional supplemental products generated from nutritional supplement platform 202 after an initial purchase (which for example occurred following the analysis of genomic and/or blood serum data). In examples, data provided by the user may be re-analysed (for example, physically tested by a lab and/or fed back through nutritional supplement platform 202) to offer more accurate recommendations and ingredient profiles, for example in cases where the dataset initially provided may not suffice for the requested product.

In examples, genomic data and blood serum data may be pre-stored in genomic data storage 232 and blood serum data storage 234, respectively. In examples, samples must be taken prior to any recommendations being made by nutritional supplement platform 202. In some examples, samples may be stored in the form of both digital formats and/or stored as physical specimens in a biobank which can later be re-analyzed by a laboratory worker.

Whenever a nutritional supplement plan is to be implemented for the user, genomic data and blood serum data may be retrieved from genomic data storage 232 and blood serum data storage 234, respectively.

According to an implementation, data analyzer 224 may be configured to receive one or more of qualitative data or quantitative data which may include but is not limited to genomic data, blood serum data, physiological data, and well-being data of the user. In an implementation, physiological data and well-being data of the user may be pre-stored in physiological data storage 236 and well-being data storage 238, respectively. In some implementations, data analyzer 224 may obtain physiological data and well-being data of the user from physiological data storage 236 and well-being data storage 238, respectively. According to an implementation, physiological data and well-being data of the user may be obtained from qualitative data sources, such as individualized survey data which may be stored in survey data storage 242 and/or from data sources 206-(1-T). In an implementation, data analyzer 224 may be configured to analyze genomic data, blood serum data, physiological data, and well-being data of the user.

According to an implementation, data analyzer 224 or model training engine 226 may be configured to construct a nutritional supplement model. In some examples, the nutritional supplement model may be constructed based on an artificial intelligence model. In examples, the nutritional supplement recommendation and subsequent provision may require the user to provide data in one or multiple formats before data analyzer 224 or model training engine 226 is configured to construct a nutritional supplement model. In examples, the ingredient profile is predetermined to varying extents, for example because of the clinical data justifying the use of certain ingredients in a specific application, however the dosage by which the nutritional supplement is provided may be dependent upon the dataset offered up by the user.

In examples, an output of the nutritional supplement model may include formulation for a nutritional supplement to be ingested by the user, for example the physical or actual or constitutional formulation of a supplement product for consumption by the user. In examples, the nutritional supplement may include a plurality of ingredients. According to an implementation, data analyzer 224 or model training engine 226 may be configured to construct the nutritional supplement model for achieving optimum levels of blood serum markers for a user for a performance goal of the user, for example based on scientific research that defines optimum levels of blood serum markers. According to an implementation, data analyzer 224 or model training engine 226 may be configured to construct the nutritional supplement model for achieving optimized performance or well-being of the user, for example as may be determined by the user reaching or achieving one or more performance goals of the user, or as may be determined by the responses to a user survey. In examples, optimum levels of blood serum markers may be determined considering at least health information related to the user (for example any conditions that the user has, blood pressure, cholesterol levels, and the like), demographic information related to the user (for example age, physical sex, racial background, and the like), and/or physiological information related to the user (for example height, weight, activity level, etc. In examples, optimum levels of blood serum markers for the user may be dependent on one or more performance goals of the user. For example, optimum levels of blood serum markers for a user with a performance goal related to long distance running may be different from optimum levels of blood serum markers for a user with a performance goal related to swimming or a performance goal related to running at a sprinting pace and distance. In an example, a nutritional supplement formulation may be a combination of ingredients that make up a nutritional supplement. In examples, a nutritional supplement formulation may be determined by or be impacted or influenced by factors that are specific to a users, including but not limited to factors such as biological gender, height, weight, and/or blood serum markers. In examples, the nutritional supplement formulation may be a product that may be used by a user as a supplement to a normal diet and the nutritional supplement may be a source of macronutrients and/or micronutrients. In examples, a nutritional supplement may be formulated and/or used to address or treat a nutritional deficiency, to maintain an adequate or targeted intake of certain nutrients, or to support specific physiological functions. In examples, a nutritional supplement may be taken in oral form and may be formulated and delivered as a dose.

In examples, an ingredient may be defined as a constituent part of a nutritional supplement. An ingredient may also be referred to as a dietary ingredient. In examples, an ingredient may comprise a macronutrient, a micronutrient, or a combination of macronutrient and micronutrient. Macronutrients are defined as a class of chemical compounds that humans consume in relatively large quantities, and which provide humans with energy. There are three principal classes of macronutrients: carbohydrate, protein, and fat. In examples, a number of ingredients in various measures or quantities may together make up a nutritional supplement. Micronutrients are defined as a class of chemical compounds that humans consume in relatively small amounts, such as vitamins and minerals. Micronutrients are vitamins and minerals needed by the body in small amounts (relative to macronutrients). They play a critical role in the function of the body and overall health and deficiency in a micronutrient can often result in detrimental, severe, and/or life-threatening conditions. They perform a range of functions, including enabling the body to produce enzymes, hormones and other substances needed for normal growth and development. Examples of micronutrients are calcium, iron, vitamin C, etc.

According to an implementation, model training engine 226 may be configured to train a nutritional supplement model, for example using an artificial intelligence model, to improve correlations in the nutritional supplement model. In an example, data received from or related to a user (for example, athletic performance data, subjective feedback, and/or blood serum sample data) can be used by model training engine 226 to make additional associations between a user's genome and their outcomes, based on the initial provision of the nutritional supplement and its subsequent success of implementation. In examples, dosages and compounds may be adjusted or adapted for the user and for one or more future users if the association is determined to be strong or significant. For example, in some cases a user may have a specific genotype that has previously not been associated with better iron absorption in clinical literature, but that seems to be associated in improved performance and/or blood serum quantities of iron. In this case, model training engine 226 may use that information and apply it to the artificial intelligence model to increase/reduce the iron quantity for other users with the same or similar genotype/profile.

In examples, the artificial intelligence model is a machine learning model incorporating data sets associated with a population (i.e., a set of one or more) of users. In examples, the artificial intelligence model may be selected from the group of artificial intelligence model types consisting of a linear regression model, a logistic regression model, a polynomial regression model, a linear discriminant analysis model, a decision tree model, a naïve bayes model, a K-nearest neighbor model, a learning vector quantization model, a support vector machine, a bagging and random forest model, and a deep neural network. An examples by which a nutritional supplement model may be constructed is described in detail in the description of FIG. 2B.

Referring back to FIG. 2A, according to an implementation, model updation engine 228 may be configured to iteratively update the nutritional supplement model based on one or more of actual levels of blood serum markers, genomic data (which may be original genomic data or updated genomic data), blood serum data (in examples, blood serum data is the output of the initial analysis of blood serum markers in a blood serum sample), physiological data, and well-being data of the user after the user ingests the nutritional supplement. According to some embodiments, blood analysis and user survey information (for example, to assess user well-being and differences in performance levels or progression towards or away from performance goals) may be repeated on a periodic basis. In an example, blood serum marker testing on a user may be at a periodic interval, for example blood serum marker testing may be performed every month or once every three months on the user to generate updated blood serum data of the user. In some examples, user survey information may be obtained periodically, for example after every month or every three months, the survey may be repeated and updated survey data may be obtained. The survey data may include subjective information about the user such as responses to an increase or decrease in nutritional supplements or a change in formulation of nutritional supplements. For example, an increase of a nutritional supplement may result in a response of the user of nausea or fatigue or may result in a response of the user as obtaining better performance in achieving a goal. In examples, updated blood serum marker levels for the user and optimum levels of blood serum markers may be used together with user survey data to determine the user's unique response to a nutritional supplement derived from a nutritional supplement model. Model updation engine 228 may adjust the nutritional supplement based on the user's unique response, for example in relation to an updated nutritional supplement resulting in a user achieving closer to the optimum levels of blood serum markers, and/or resulting in improved physical performance of the user, and/or resulting in a user survey demonstrating a user perceived benefit as determined by qualitative/quantitative user survey data.

In examples, the degree or rate of absorption of nutrients in a nutritional supplement varies between users, and as a result updated blood serum marker levels may be different for different users despite providing the same nutritional supplement formulation to the different users. In examples, updated blood serum marker levels may be compared to the optimum levels of blood serum markers for the user, and delta blood serum marker levels for the user may be determined. Based on the delta blood serum marker levels of the user, model updation engine 228 may determine an adjustable weighting factor for one or more ingredient in the nutritional supplement formulation. In examples, model updation engine 228 may update the nutritional supplement model in response to a weighting factor for one or more ingredients in the nutritional supplement formulation being adjusted.

According to an implementation, gamification engine 230 may implement gamification mechanisms to incentivize users to provide user survey data and/or user performance data. In an implementation, gamification engine 230 may incorporate social engineering with the gamification mechanisms to incentivize the user to utilize or participate in or engage with the gamification mechanisms. According to an implementation, gamification engine 230 may promote user engagement with the nutritional supplement platform 202 and/or promote user compliance to the program. In examples, to encourage and reward user program compliance and to recognize progression towards user performance goals, one method gamification engine 230 may employ to incentivize the user may be based on comparisons between the user and one or more other users, for example but not limited to user peer groups. In some embodiments, gamification engine 230 may employ various techniques such as leaderboards, badges, and achievement levels to incentivize the user.

In some embodiments, nutritional supplement platform 202 may include genomic data storage 232, blood serum data storage 234, physiological data storage 236, well-being data storage 238, nutritional supplement plans storage 240, and survey data storage 242. In an implementation, genomic data storage 232 may store genomic data of the users. In examples, the genomic data may be deoxyribonucleic acid (DNA) data. In an implementation, blood serum data storage 234 may store blood serum data of the users, where the blood serum data is the result of the analysis of blood serum samples. For example, blood serum data that may result from analysis of blood serum samples includes levels of 25-hydroxyvitamin D, magnesium (serum), active B12, Ferritin, the Total Iron Binding Concentration (TIBC), and/or the Full Blood Count (FBC). In an implementation, physiological data storage 236 may store physiological data of the users. In examples, the physiological data may include, but is not limited to heart rate, blood glucose level, blood pressure, respiration rate, and body temperature. In an implementation, well-being data storage 238 may store well-being data of the users. In an example, the well-being data may be related to weight, lifestyle factors, and activity level of the users. In an example, the activity level of a user may be classified as “high”, “moderate”, or “low”. In an implementation, nutritional supplement plans storage 240 may store nutritional supplement plans for users. In an example, a nutritional supplement plan may refer to the development of a single supplement product (in the form of capsules, tablets, soft gels, powders, liquids, and energy bars, or otherwise) or multiple supplement products that are recommended for a user based on the information and data provided by the user. In an implementation, survey data storage 242 may store survey data of one or more surveys provided to one or more users. In examples, survey data of a survey may include results that are obtained from gathering responses from one or more users who took a survey. In examples, a survey may be used in order to solicit user feedback regarding perceived symptoms, health information, preferences, and various other aspects. Examples of other aspects include lifestyle factors such as exercise and activity level, sport selection, environmental factors like geography/location, ethnicity, pre-existing injuries, allergies, sensitivities, what supplements the user is already taking, perceptions of supplement products/market, or athletic or performance goals. In examples, a survey may include a plurality of questions that may be asked to a user. In examples, the plurality of questions may solicit responses (or answers) from the user to the questions. In examples, a user survey may be provided electronically to a user and user responses may be collected electronically from a user. In examples, user responses to one or more questions of a user survey may be provided to nutritional supplement model, for example by model training engine 226 or model updation engine 228. In examples, user genomic data stored in genomic data storage 232, user blood serum data stored in blood serum data storage 234, user physiological data stored in physiological data storage 236, user well-being data stored in well-being data storage 238, user nutritional supplement plans stored in nutritional supplement plans storage 240, and user survey data stored in survey data storage 242 may be periodically or dynamically updated as required.

Referring again to FIG. 2A, in one or more embodiments, user device 204-(1-S) may be any device used by a user (all devices of user device 204-(1-S) are subsequently referred to as user device 204-1, however, the description may be generalized to any of user device 204-(1-S)). User device 204-1 may be a computing device, such as a desktop computer, a laptop, a tablet computer, a mobile device, a Personal Digital Assistant (PDA), or any other computing device. In an implementation, user device 204-1 may be a device, such as client device 102 shown in FIG. 1A and FIG. 1B. User device 204-1 may be implemented by a device, such as computing device 100 shown in FIG. 1C and FIG. 1D. According to some embodiments, user device 204-1 may include processor 256-1 and memory 258-1. In an example, processor 256-1 and memory 258-1 of user device 204-1 may be CPU 121 and main memory 122, respectively, as shown in FIG. 1C and FIG. 1D. User device 204-1 may also include user interface 260-1, such as a keyboard, a mouse, a touch screen, a haptic sensor, a voice-based input unit, or any other appropriate user interface. It shall be appreciated that such components of user device 204-1 may correspond to similar components of computing device 100 in FIG. 1C and FIG. 1D, such as keyboard 126, pointing device 127, I/O devices 130a-n and display devices 124a-n. User device 204-1 may also include display 262-1, such as a screen, a monitor connected to the device in any manner, or any other appropriate display, which may correspond to similar components of computing device 100, for example display devices 124a-n. In an implementation, user device 204-1 may display content for the user using display 262-1 and may accept user interactions via user interface 260-1, for example but not limited to responsive to displayed content.

Referring again to FIG. 2A, in some embodiments, user device 204-1 may include platform client 264-1. In one example, platform client 264-1 may be a thin client or cloud-based application that can be accessed over network 250 without being installed on user device 204-1. In an implementation, platform client 264-1 may be any application capable of connecting with nutritional supplement platform 202, for example to exchange data, may perform one or more defined process in or associated with user device 204-1, and may output information obtained from nutritional supplement platform 202. In an example, platform client 264-1 may facilitate a user to create, receive, organize, and otherwise manage personal data and user nutrition plans. In an implementation, platform client 264-1 may be an application that runs on user device 204-1. In some implementations, platform client 264-1 may be an application that runs on a remote server or on a cloud implementation and is accessed by a web browser. In an example, a user of user device 204-1 may be required to download and install platform client 264-1 on user device 204-1. In an example, platform client 264-1 may be provided as a default application by some application distribution and/or mobile phone platforms. In some examples, a user of user device 204-1 may select, purchase and/or download platform client 264-1 through an application distribution platform. Other user devices 204-(2-S) may be similar to user device 204-1.

In an implementation, data sources 206-(1-T) may be sources that include scientific research data, clinical research data, and other miscellaneous data. In examples, data sources 206-(1-T) may maintain data related to the users. In examples, the data may include user data records of the users, which for example may include user attributes of the users. In some examples, one or more data sources 206-(1-T) may share information with one or more different data sources 206-(1-T). In examples, one or more data sources 206-(1-T) may be isolated or standalone. In an implementation, data from one or more data sources 206-(1-T) may be provided as an input to nutritional supplement platform 202. In examples, nutritional supplement platform 202 may use data retrieved from one or more data sources 206-(1-T) to create, update, adapt, modify, or implement nutritional supplement plans for users.

Referring to FIG. 2B, in a general overview, FIG. 2B depicts a block flow diagram for determination of formulation for a nutritional supplement (also referred to as nutritional supplement formulation), according to one or more embodiments. For ease of explanation and understanding, the description provided in the present disclosure is with reference to a single user, however, the description is equally applicable to more than one user. In examples, each user may be associated with a respective nutritional supplement model (or individual nutritional supplement model).

In examples, nutritional supplement platform 202 may use an artificial intelligence model to develop personalized nutritional supplement formulation for a user. According to an implementation, nutritional supplement platform 202 may receive one or more of qualitative data or quantitative data, such as survey data 272, genomic data 274, and blood serum data 276. In an implementation, nutritional supplement platform 202 may determine nutritional supplement formulation 288 based on survey data 272, genomic data 274, blood serum data 276, and one or more potential ingredients, shown in FIG. 2B as ingredient 278, ingredient 280, ingredient 282, ingredient 284, and ingredient 286. FIG. 2B illustrates five ingredients, however the number of ingredients may be greater than or less than five. In examples, a given nutritional supplement formulation 288 may include one or more of potential ingredients 278-286.

According to an implementation, nutritional supplement platform 202 may generate digital insights report 290. In examples, digital insights report 290 may include key insights obtained from genomic data 274 and blood serum data 276. In examples, digital insights report 290 may be a user health report and/or a user nutrition report. In some examples, digital insights report 290 may indicate progress of a user in relation to both nutritional (for example, as defined by blood serum data 276) and training goals provided by or derived from user survey data 272. In examples, digital insights report 290 may feature genetic information of the user in the form of specific ‘effects’ in both the realm of nutrition or training/injury predispositions, for example nutrition information (for example, vitamin A requirement, vitamin B6 requirement, vitamin B12 requirement, antioxidant requirement, caffeine sensitivity, salt sensitivity, carbohydrate sensitivity, fat sensitivity, etc.), and/or training information (for example, ACL injury risk, Achilles injury risk, lower back injury risk, endurance response, aerobic VO2 max trainability, recovery efficiency, strength response, power response, etc.). In examples whereby blood sampling has taken place, digital insights report 290 may also include information pertaining to the current nutritional status of the user, such as the quantities of the tested biomarker residing in the user's blood serum at the time of testing and the association with the user's health, wellbeing, and performance.

According to some implementations, nutritional supplement platform 202 may incorporate feedback model 292 to adjust the nutritional supplement formulation with the purpose of attempting optimize performance and well-being of the user. In an implementation, feedback model 292 may perform on or more feedback cycles for reformulation of the nutritional supplement, for example on a periodic or aperiodic basis. In examples, survey data 272 and blood serum data 276 of one or more users may be updated on a periodic or aperiodic basis. For example, survey data 272 and blood serum data 276 may be updated every twelve weeks or every three months, or on request of the user. In an implementation, feedback model 292 may incorporate updated survey data and updated blood serum data in the reformulation of the nutritional supplement. In examples, the nutritional supplement may be reformulated on a periodic or aperiodic basis in light of the information reflecting impact of the current nutritional supplement formulation obtained through the one or more feedback cycles which provide updated information on the user's performance and/or well-being. Examples by which nutritional supplement formulation and reformulation may be determined are described in detail below.

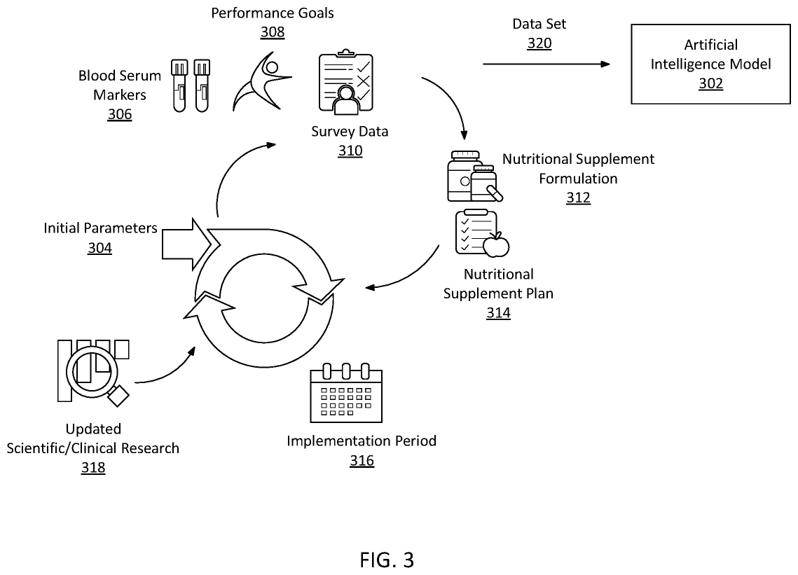

FIG. 3 depicts a representation of an example of nutritional supplement model 300, according to one or more embodiments. In examples, the purpose of nutritional supplement model 300 may be to achieve optimum levels of blood serum markers for a user “N” by formulation of a nutritional supplement to be ingested by the user “N”. In examples, the purpose of nutritional supplement model 300 may be to achieve optimized performance or well-being of a user “N” by formulation of a nutritional supplement to be ingested by the user “N”. In an implementation, nutritional supplement model 300 may be trained via artificial intelligence model 302.

According to an implementation, initial parameters 304 may be fed into nutritional supplement model 300. In an example, initial parameters 304 may include personal characteristics of user “N” that do not change over time (or change deterministically over time). In examples, initial parameters 304 may include genomic data, blood serum data, physiological data, and well-being data of user “N”. Examples of initial parameters 304 include, but are not limited to, height, biological gender, age, ethnicity, weight, activity level, sports participated in, lifestyle factors (such as smoking or drinking), and pre-existing conditions. According to some implementations, blood serum markers 306 and/or performance goals 308 of user “N” may also be fed into nutritional supplement model 300. In an implementation, a blood serum marker level (which may also be referred to a blood serum marker concentration) may be associated with one or more nutritional supplement ingredients. In examples, blood testing outcomes are reported as the overall proportion of the blood serum marker per mL or dL of blood.

In some examples, it may not be possible or may be impractical or may be difficult to test for the actual blood serum marker, for example vitamin B2 (B2), vitamin B6 (B6), vitamin D (D), iron (Fe), and potassium (K). In examples, the testing methodology may use a proxy or a metabolite of the original nutritional supplement ingredient to assess the overall absorption, metabolism, and/or presence of the nutritional supplement ingredient.

In examples, the measured or actual level of one or more blood serum markers for user “N” may be represented by the notation, “(Marker Name) N”. Accordingly, the actual levels of blood serum markers for user “N” according to the above list may be represented as B2N, B6N, DN, FeN, and KN. Further, in examples, the optimum levels of blood serum markers for the user “N” may be represented as B2N-O, B6N-O, DN-O, FeN-O, and KN-O.

According to an implementation, the optimum levels of blood serum markers may be determined empirically through scientific/clinical research or other information. In some implementations, the optimum levels of one or more blood serum markers may be determined based on an output from artificial intelligence model 302. In some implementations, the optimum levels of one or more blood serum markers may be based on one or more initial parameters 304. In examples, the optimum levels of one or more blood serum markers may be dependent on one or more performance goals 308 of user “N” and/or biological gender of user “N”. In some examples, the optimum levels of one or more blood serum markers may be dependent on a measurement of body mass index (BMI) of user “N”. In examples, there may be one or more sets of optimum levels of blood serum markers for males and there may be one or more sets of optimum levels of blood serum markers for females. In some examples, the optimum levels of one or more blood serum markers for user “N” may be dependent on one or more performance goals 308 of user “N”.

In some embodiments, the actual levels of one or more blood serum markers for user “N” may be compared to the optimum levels of the one or more blood serum markers to obtain a difference between the actual levels of blood serum markers and the optimum levels of blood serum markers for user “N”. In an example, the absolute value and/or the change in value of one or more blood serum markers for user “N” may be used to update nutritional supplement model 300. In examples, the difference between the actual levels of one or more blood serum markers for user “N” and the optimum levels of the one or more blood serum markers for user “N” may be referred to as delta blood serum marker levels for user “N”. In examples, the delta blood serum marker levels may be expressed as follows:

$$Δ B {2}_{N} = B {2}_{N} - B {2}_{N - O} \qquad ( 1 )$$

$$Δ B {6}_{N} = B {6}_{N} - B {6}_{N - O} \qquad ( 2 )$$

$$Δ {D}_{N} = {D}_{N} - {D}_{N - O} \qquad ( 3 )$$

$$Δ {Fe}_{N} = {Fe}_{N} - {Fe}_{N - O} \qquad ( 4 )$$

$$Δ {K}_{N} = {K}_{N} - {K}_{N - O} \qquad ( 5 )$$

According to an implementation, a user survey may be provided to user “N”. In an implementation, one or more responses to the survey may be collected from the user “N”. In an example, the survey may include one or more questions. In examples, the one or more questions may solicit responses or answers from user “N” to the one or more questions. In an example, survey questions and/or survey answers may be of various formats. In examples, a survey question may be accompanied by choice of numeric answers in a range of 1 to 5, where 1 is indicative of poor and 5 is indicative of good. In some examples, a survey question may solicit Yes/No responses (answers) from user “N” to the question. In examples, “Yes” or “No” answers may be codified into values. For example, “Yes” may be assigned a value of 1 and “No” may be assigned a value of 0. In some examples, a question may be accompanied by a selectable list of standard or predetermined answers, for example in a multiple choice format. In an implementation, one or more responses received from user “N” in response to one or more questions may be processed to determine survey data 310. In an implementation, survey data 310 may be provided to nutritional supplement model 300. In examples, survey data 310 may enable subjective changes associated with user “N” to be captured in a quantitative and/or qualitative manner. Examples of survey questions and potential answers are given in the Table below.

TABLE 2

| Describe the level of bloating that you have experienced in the last period | |
|---|---|
| compared to the previous period. | |
| Much less □ Somewhat less □ The same □ Somewhat more □ Much more □ | |
| Please indicate whether you have experienced any of the following symptoms | |
| in the last period by marking “YES” or “NO” beside each symptom. | |
| Abnormal rapid heartbeat | YES □ NO □ |
| Stomach pain | YES □ NO □ |
| Dizziness or confusion | YES □ NO □ |
| Tremors or shaking | YES □ NO □ |
| Numbness or tingling of arms or legs | YES □ NO □ |
| Loss of consciousness | YES □ NO □ |

Please rate the quality of your sleep in the last period on a scale of 1 to 5,
where 1 is poor and 5 is good.
1 □ 2 □ 3 □ 4 □ 5 □
Over the last period, how often have you felt nervous, anxious, or on edge?
Not at all □ Several days □ More days than not □ Nearly every day □
Over the last period, have you experienced any change in appetite?
More hungry/Hungry more often □ Less hungry/Hungry less often □ No change □

According to an implementation, nutritional supplement formulation 312 (i.e., formulation for a nutritional supplement) for user “N” may be determined as a function of initial parameters 304, delta blood serum marker levels, and survey data 310 obtained from user “N”. In an example implementation, nutritional supplement formulation 312 may be mathematically represented using Equation (6) provided below.

$${S}_{N} = f ( initial {parameters}_{N} , Δ blood serum {markers}_{N} , survey {data}_{N} ) \qquad ( 6 )$$

where S_{N} represents nutritional supplement formulation, initial parameters_{N} represents initial parameters for user “N”, Δblood serum markers_{N} represents delta blood serum marker levels for user “N”, and survey data_{N} represents the survey data obtained from user “N”.

In examples, nutritional supplement formulation 312 may be represented as a combination of one or more unique ingredients. In an implementation, the concentration of each of the one or more ingredients may be determined based on initial parameters 304 and blood serum markers 306. In examples, the concentration of each of the one or more ingredients may be adjusted based on factors such as the user's physiological ability to absorb the ingredient. In examples, the ability to absorb the ingredient may be a consequence of the genetic data of the user provided to data analyzer 224. For example, certain SNPs and genes allow data analyzer 224 to determine the overall effectiveness of absorption that a user may be predisposed to. In examples, data analyzer 224 may use population data from clinical research to inform and calculate the expected change or quantity of blood serum markers in the blood based on the dosage provided. In examples, data analyzer 224 may make inferences as to a user's ability to absorb an ingredient based on subsequent blood serum testing data. In examples, clinical research may indicate that certain lifestyle factors or ethnicity or environmental factors may affect a user's ability to absorb a certain nutrient. In examples, data analyzer 224 may adjust a supplement profile based on data provided in the form of survey information.

In some examples, the ingredients of nutritional supplement formulation 312 may align with blood serum markers 306. In examples, there may be more or less ingredients of nutritional supplement formulation 312 than blood serum markers 306, that is, there may not be a one to one relationship between ingredients of nutritional supplement formulation. In an implementation, data analyzer 224 may associate adjustable weighting factors with ingredient terms in nutritional supplement model 300 determinations based on nutritional supplement ingredient absorption rates of user “N”. In examples, the quantity of an ingredient provided in the nutritional supplement formulation for a user may be informed by blood serum data provided by the user for specific blood serum markers (for example, if the user's vitamin D level is already sufficient, the dosage of vitamin D will be reduced to align with the blood serum marker of the user).

In examples, potential ingredients in nutritional supplement formulation 312 may be numbered from 1 to M. In an example, the potential ingredients may be represented by the notation “IM” where M is the ingredient number. In some examples, nutritional supplement formulation 312 for user “N” may be expressed using Equation (7) provided below.

$${S}_{N} = {Σ}_{1}^{M} {α}_{M} \cdot {I}_{M} , \qquad ( 7 )$$

where α_{M} represents an adjustable weighting factor for ingredient M.

In some embodiments, the analysis of relevant SNPs may reveal that user “N” has a low absorption rate for one of the ingredients in nutritional supplement formulation 312. In an example, one or more relevant SNPs may reveal that user “N” has a lower absorption rate for ingredient “IM”. In such a case, the value of the weighting factor “αM” for user “N” may be increased to offset the lower absorption rate.

In examples, initial parameters 304 may be used to adjust the adjustable weighting factors “αM” based on prior information (for example, from scientific and/or clinical research or from artificial intelligence model 302) regarding relationships between initial parameters 304 and nutritional supplement ingredient absorption rates. For example, it may be known that white European men with a high activity level tend to have a low absorption rate of iron. Accordingly, the adjustable weighting factor for iron for user “N” with initial parameters 304 including “white European”, “male”, and “high activity level” may be increased to account for this low absorption level.

According to an implementation, upper limits and lower limits may be imposed on amounts of one or more ingredients included in nutritional supplement model 300. In examples, one or more ingredients in the nutritional supplement may be subject to an upper limit or a lower limit. In examples, in nutritional supplement formulation 312, if the nutritional supplement contains an ingredient for which the adjustable weighting factor and the amount of the ingredient in combination exceeds the upper limit for the ingredient, then the amount of the ingredient in the nutritional supplement may be set to the upper limit. In examples, in nutritional supplement formulation 312, if the nutritional supplement contains an ingredient for which the adjustable weighting factor and the amount of an ingredient in combination is less than a lower limit for the ingredient, then the amount of the ingredient in the nutritional supplement is set to the lower limit. In examples, if the nutritional supplement formulation calls for a weighted amount of an ingredient that exceeds or is below the maximum or minimum level for the ingredient respectively, the relative proportions of one or more ingredients in the nutritional supplement formulation is first determined and then the ingredient amounts are proportionally scaled such that no one ingredient exceeds or falls below and maximum or minimum level for the ingredient. Accordingly, nutritional supplement model 300 ensures that limits on the concentration of an ingredient are not exceeded and that the resultant nutritional supplement is therefore safe to consume. According to an implementation, nutritional supplement plan 314 may be determined for user “N”. According to some implementations, implementation period 316 for nutritional supplement plan 314 may also be determined.

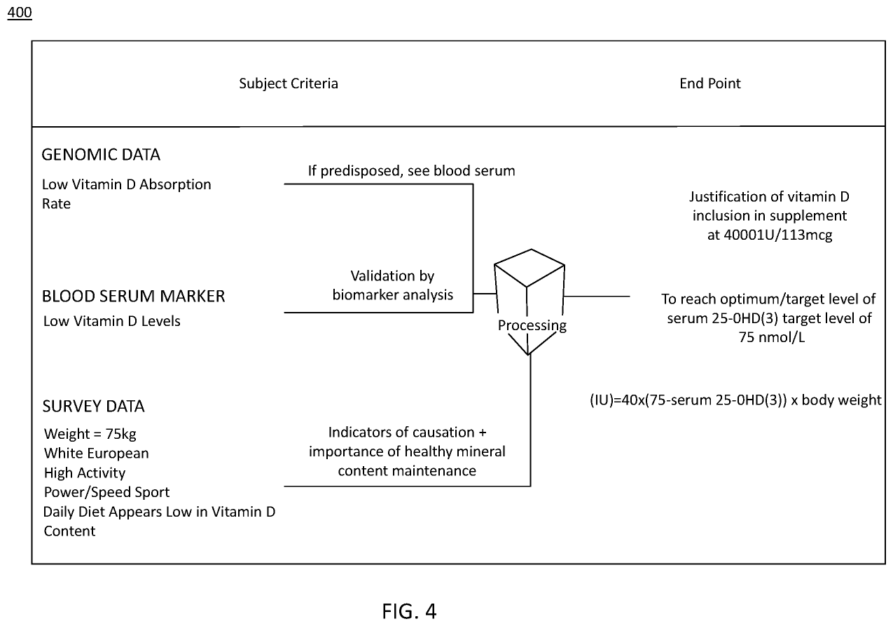

FIG. 4 illustrates example 400 of formulation for a nutritional supplement prepared for use the user “N”, according to one or more embodiments.

In the example of FIG. 4, use of genomic data 274, blood serum markers 306 retrieved or determined by blood serum data 276, which is digitized information that results from analysis of a blood serum sample, and/or and survey data 272/310 to determine the formulation of a nutritional supplement is described. In examples, genomic data 274 identifies that user “N” has low vitamin D absorption rate, and blood serum markers 306 identifies that the user “N” has low vitamin D levels. Further, survey data 310 includes “weight=75 kg”, “white European”, “high activity level”, “power/speed sport”, “daily diet appears low in vitamin D content”. In examples, the amount of vitamin D to be included in the nutritional supplement may then be determined based on the genomic data, blood serum markers 306, and survey data 310. In the example shown in FIG. 4, to reach the optimum level of blood serum marker (i.e., 25-OHD(3), 5000 IU/113 mcg vitamin D is required by the user “N”. As illustrated in FIG. 4, “if predisposed, see blood serum” indicates that the genetic data for the user has indicated that the user is predisposed to a low vitamin D absorption and therefore nutritional supplement platform 202 will validate that the blood serum data of the user also indicates such an outcome. As illustrated in FIG. 4, “validated by biomarker analysis” indicates that the next step f is that nutritional supplement platform 202 validates that the user does have a lower vitamin D level in the blood than is otherwise deemed as optimal (in this user's scenario). As illustrated in FIG. 4, “indicators of causation+importance of healthy mineral content maintenance” indicates that it is likely the user's genetics are a potential cause of the low vitamin quantity in the blood serum markers, for example due to poor absorption. The information based on the indications may be provided to the user to explain to the user why it is important to maintain an optimal or higher level of the nutritional supplement ingredient.

“Processing”—this refers to the ‘work’ done by nutritional supplement platform 202 and machine in combining, analyzing, and calculating the total weighting, score, and subsequent dosage to be provided in the supplement product(s).

“Justification of vitamin D inclusion in supplement at 4000 IU/113 mcg to reach optimum/target level of serum 25-UHD(3) target level of 75 nmol/L”

(IU)=40×(75-serum 25-OHD(3))×body weight”—is a scientifically reasoned and referenced calculation of the exact dosage required for this user to reach the optimal quantity of the product in the blood serum.

According to some embodiments, blood serum marker testing and survey information gathering may be repeated on a periodic or aperiodic basis. In an example, after every three months, a blood serum marker testing may be performed on the user to generate updated blood serum data of the user. In some examples, after every three months, a user survey may be provided and updated survey data of the user may be obtained. This period between a previous measurement and a current measurement may be referred to as a “measurement interval”. In examples, updated blood serum marker levels for the user and optimum levels of blood serum markers for the user may be used together with survey data to determine the user's unique response to the nutritional supplement derived from the nutritional supplement model. In examples, the formulation of the nutritional supplement may be adjusted (for example through adaptation by or of the nutritional supplement model) based on the user's response to the nutritional supplement to improve the performance of the nutritional supplement in relation to its ability to better achieve optimum levels of blood serum markers, to improve the physical performance of the user, and to improve the perceived benefits as determined by qualitative/quantitative survey data. In examples, some users may absorb nutrients in nutritional supplements more efficiently than other users. The updated blood serum marker level may be compared to the optimum levels of blood serum markers for the user, and delta blood serum marker levels may be determined. In examples, delta blood serum marker levels may be different for different users even if the different users were provided with the exact same nutritional supplement formulation.

In examples, the blood serum marker levels gathered from the user may be referred to as “result” and the optimum levels of blood serum markers may be referred to as “target”. Further, a measurement interval be represented by “T”. In examples, initial blood serum marker levels are taken at “T0” and are referred to as “result0”. In examples, blood serum marker levels collected at the end of a first measurement interval “T1” are referred to as “result1”. According to an implementation, model updation engine 228 may measure blood serum marker levels for each ingredient in a nutritional supplement at the end of the measurement interval T+1, providing delta blood serum marker levels according to (result_{T+1}−result_{T}) and may compare the delta blood serum marker level value or values to zero. In examples, delta blood serum marker level values approaching zero may mean that the optimum levels of blood serum markers for the user have been reached. In some implementations, model updation engine 228 may measure, for each ingredient, at the end of the measurement interval T+1, (result_{T+1}−result_{T})−(result_{T}−result_{T-1}), representing a rate of change or a step change of the delta blood serum marker level value or values and may compare this to the expected step change over the measurement interval. In examples, measurements of the delta blood serum marker level values or of the rate of change or a step change of the delta blood serum marker level value or values may indicate that a change in the ingredient in the nutritional supplement takes a longer period of time to impact the blood serum marker levels than one measurement interval.

In examples, if for a blood serum marker, (result_{T+1}−result_{T})>0 (i.e., the optimum levels of the blood serum marker has not been reached), then the amount of the ingredient associated with the blood serum marker (referred to as the current ingredient in this example) in the nutritional supplement may be adjusted (for example, increased), as this may be an indication that the user has less than normal ability to absorb the ingredient. In examples, if (result_{T+1}−result_{T})>0, then an additional ingredient that improves the absorption of the current ingredient may be added to the nutritional supplement formulation. In some examples, if (result_{T+1}−result_{T})<0 (i.e., the optimum levels of blood serum markers have been exceeded), then the amount of the current ingredient may be adjusted (for example, decreased), as this may be an indication that the user has higher than normal ability to absorb the ingredient.

Considering, for example, for a current ingredient that the rate of change (step change) of the delta blood serum marker level (result_{T+1}−result_{T})−(result_{T}−result_{T-1}), if the step change is less than the expected step change that is associated with the change in the amount of the ingredient over the measurement interval (i.e., the user may be converging to the optimum levels of blood serum markers associated with the current ingredient less quickly than is expected), then the amount of the current ingredient in the nutritional supplement formulation may be adjusted (for example, increased) as this may be an indication that the user has less than normal ability to absorb the current ingredient. Further, if the step change is greater than the expected step change that is associated with the change in the amount of the ingredient over the measurement interval for the current ingredient (i.e., the user may be converging to the optimum levels of blood serum markers associated with the current ingredient more quickly than is expected), then the amount of the current ingredient in the nutritional supplement formulation may be adjusted (for example, decreased), as this may indicate that the user has higher than normal ability to absorb the current ingredient.

In some examples, if the delta blood serum marker level for a current ingredient (result_{T+1}−result_{T})>0, then the adjustable weighting factor of the current ingredient in the nutritional supplement may be adjusted (for example, increased), as this may be an indication that the user has less than normal ability to absorb the current ingredient. In examples, if the delta blood serum marker level for a current ingredient (result_{T+1}−result_{T})<0, then the adjustable weighting factor for the current ingredient may be adjusted (for example, decreased), as this may be an indication that the user has higher than normal ability to absorb the current ingredient.

In examples, again considering the rate of change (step change) of the delta blood serum marker level of a current ingredient, (result_{T+1}−result_{T})−(result_{T}−result_{T-1}), if the step change is less than the expected step change of the current ingredient that is associated with the change in the amount of the ingredient over the measurement interval, then the adjustable weighting factor for the current ingredient may be adjusted (for example, increased), as this may be an indication that the user has less than normal ability to absorb the current ingredient. Further, if the step change of the delta blood serum marker level of a current ingredient is greater than the expected step change of the current ingredient that is associated with the change in the amount of the ingredient over the measurement interval (i.e., the user may be converging to the optimum levels of blood serum markers more quickly than is expected), then the adjustable weighting factor for the current ingredient may be adjusted (for example, decreased), as this may indicate that the user has higher than normal ability to absorb the current ingredient. In examples, by adjusting the adjustable weighting factors for the user instead of adjusting the absolute amount of the ingredient, the same baseline dosing formulation can be used for multiple users. This allows the baseline dosing formulation to be adjusted by the artificial intelligence model and such changes to the baseline dosing formulation to be readily applied to multiple users where relevant. In examples, a baseline dosing formulation is a nutritional supplement formulation that is determined based solely on a user's blood analysis, genomic data analysis, and survey responses, before the user begins taking the nutritional supplement. This is in contrast to a reformulation of a nutritional supplement, which considers a user's blood analysis and survey responses after a measurement interval during which the user has been taking the nutritional supplement.

According to an implementation, through the regular blood analysis, reporting of physical performance metrics and reporting of subjective perceived benefits and disadvantages (for example through the use of a user survey), the expected improvement in user's performance can be tracked against the actual performance change of the user. In some examples, this may demonstrate that the user absorbs ingredients of the nutritional supplement formulation less efficiently or more efficiently than expected.

In examples, the user may input his or her physical activity (for example, through a mobile application or web interface) including physical performance metrics (for example, maximum pace, maximum distance, VO2 max, power response, endurance response, strength response, aerobic (VO2) trainability, recovery efficiency, personal records, etc.). According to an implementation, model updation engine 228 may retrieve expected physical performance metrics associated with the level of physical activity of the user and the current nutritional supplement formulation for the user (if any) and may form a comparison between the expected physical performance metrics for the user and the actual physical performance metrics of the user. The result of the comparison may be used to either adjust the adjustable weighting factors of one or more ingredients of the nutritional supplement for the user and/or adjust the absolute amount of one or more ingredients of the nutritional supplement for the user.

In examples, subjective perceived benefits submitted by the user (for example through a user survey) and for example which may be captured through a web and/or a mobile application may be compared to a database of expected benefits associated with the level of activity of a user and the current nutritional supplement formulation for the user (if any). In an implementation, model updation engine 228 may determine any difference between the expected benefits for the user and the actual perceived benefits of the user and may quantify this difference. The quantified difference result of the determination may be used to either adjust the adjustable weighting factors of one or more ingredients of the nutritional supplement and/or adjust the absolute amount of one or more ingredients of the nutritional supplement.

In examples, a comprehensive digital insight report may be provided to a user outlining key insights obtained from the analysis blood serum data to reveal or determine blood serum marker data, genomic data, and user survey data. In some examples, progress in relation to one or more of nutritional goals (as defined by blood serum data) and training goals (as provided by physical performance data) provided by or derived from the survey data.

According to an implementation, reformulation of a nutritional supplement on a periodic or aperiodic basis may consider the blood serum data analysis, genomic data analysis, performance metrics analysis and survey data analysis either individually or in combination. In some implementations, the survey data may indicate that the user has an intolerance to one or more of the ingredients of the nutritional supplement. In some examples, a diet plan or physical training plan may be provided to the user to enhance, improve absorption of, or enable the replacement of one or more of the ingredients of the nutritional supplement or to increase the rate of performance improvement towards one or more of the user's goal.

According to an implementation, nutritional supplement platform 202 may utilize data acquired from the above feedback process to notify a feedback model 292. In an example the feedback model 292 may indicate that the level of iron in the nutritional supplement formulation is too high for male athletes. Accordingly, lower doses of iron may be included in future nutritional supplement formulations for male athletes. The collection of data from multiple users may be subject to multivariate analysis, for example, through updates to the individual nutritional supplement model. In examples, feedback model 292 will leverage any insights obtained from one user or multiple users to make better-informed decisions regarding product selection and ingredient dosages (for example, if and when data may indicate that there is a more effective implementation). In examples, for example with a sufficiently large sample size, feedback model 292 may make statistically significant associations between additional genes and specific performance and/or nutritive outcomes that can then be applied to feedback model 292 through the application of machine learning or artificial intelligence systems.

According to an implementation, updates from scientific and/or clinical research may be input to the nutritional supplement model. In an implementation, the nutritional supplement model may be provided with scientific and/or clinical research updates when the scientific and/or clinical research updates become publicly available. In an example, updates from scientific and/or clinical research may be obtained from data sources 206-(1-T). In an implementation, a filtering process may be performed to determine relevant scientific and/or clinical research updates. In examples, only relevant scientific and/or clinical research updates may be provided to the nutritional supplement model. In an example, information from scientific and/or clinical research updates may be codified prior to being provided to the nutritional supplement model. In the example shown in FIG. 3, updated scientific/clinical research 318 is provided to nutritional supplement model 300. In some implementations, data set 320 of user “N” may be created from nutritional supplement model 300. In an example, data set 320 may include information about user “N”. In an implementation, data set 320 may be provided to artificial intelligence model 302 for further processing as previously described.

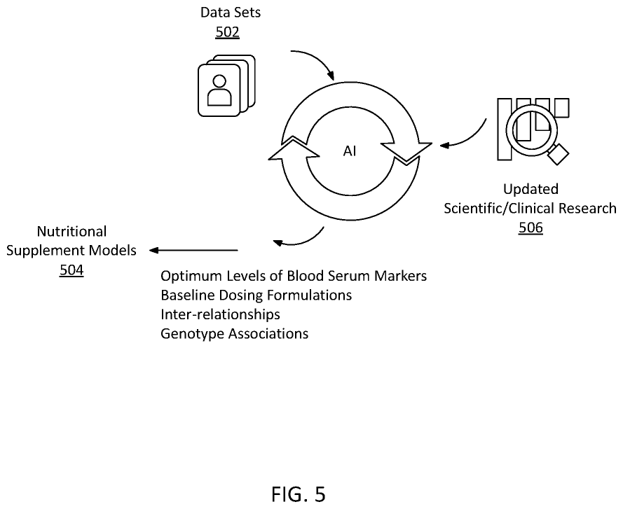

FIG. 5 illustrates artificial intelligence model 500, according to one or more embodiments. In an example, artificial intelligence model 500 may be an example of artificial intelligence model 302. In examples, artificial intelligence model 500 may be a machine learning model incorporating data sets 502 of a population (i.e., multiple users). According to an implementation, artificial intelligence model 500 may be configured to analyze and exploit data sets 502 aggregated across many users to further develop and enhance individual nutritional supplement models 504, which may be examples of nutritional supplement model 300. In an example, nutritional supplement model 300 may be trained via artificial intelligence model 500 to improve correlations in nutritional supplement model 300. In an implementation, data sets 502 created from individual nutritional supplement models 504 from many users may be analyzed by artificial intelligence model 500 to better identify relationships between SNP(s), levels of blood serum markers, and survey data. This may lead to an increased initial level of effectiveness of the nutritional supplement formulations due to the improvements in the formulation of the nutritional supplements by artificial intelligence model 500′.

In the example shown in FIG. 5, artificial intelligence model 500 may take data sets 502 as an input and process data sets 502 to determine, for example, optimum levels of blood serum markers for one or more populations of users, for example populations of users associated with one or more data sets 502, baseline dosing formulations, inter-relationships between ingredients, and genotype associations between data sets 502 to provide nutritional supplement formulations to more effectively help users achieve specific performance goals.

In an implementation, artificial intelligence model 500 may use artificial intelligence (AI) to process data sets 502. In some examples, AI may be used as a means of minimization of root mean square error between parameters. In examples, artificial intelligence model 500 may be trained with data sets 502 to determine weighting coefficients for individual nutritional supplement ingredients which best achieve the goal of minimizing the root mean square error over data sets 502. In an example, scientific and/or clinical research may be fed into artificial intelligence model 500. According to an implementation, updates from scientific and/or clinical research may be an input to artificial intelligence model 500. In an implementation, artificial intelligence model 500 may be fed with the scientific and/or clinical research updates when the scientific and/or clinical research updates become publicly available. In an example, updates from scientific and/or clinical research may be obtained from data sources 206-(1-T). In an implementation, a filtering process may be performed to determine relevant scientific and/or clinical research information or updates. In examples, a scoring method may be determined that will assess the validity of the scientific data, for example taking into consideration whether the scientific data has been peer-reviewed, whether the scientific data has been published by a reputable journal, what year was the scientific data was collected, what the sample size used in the study the collected the scientific data was, what the ethnic diversity of the studied cohort in the scientific data was, and/or whether the authors of the scientific data have conflicts of interest. The company will also have a dedicated research team that will develop and utilize such a method whilst combing through novel data that we can implement into the algorithm to inform the decision-making model.

In examples, only relevant scientific and/or clinical research updates may be provided to nutritional supplement models 504. In the example shown in FIG. 5, updated scientific/clinical research 506 is fed into artificial intelligence model 500. In an example, updated scientific/clinical research 506 may be an example of updated scientific/clinical research 318.

In examples, users may provided with an option to permit the use of their user data (which may be anonymized or only provided to nutritional supplement platform 202 in the aggregate, for example nutritional supplement platform 202 may have the ability to combine user data in a single pool of anonymized information that may be removed from content that may be personally identifiable.) to be used for improvement of artificial intelligence model 500, for example by possible improvements in the formulation of subsequent supplement profiles and may be used to identify new associations between SNPs and performance traits. In examples, AI may be used to determine inter-relationships between initial parameters and performance goals and measured characteristics.

According to some implementations, more than one artificial intelligence model 500 may be established. In examples, each of the one or more artificial intelligence models 500 may be configured to determine a subset of optimum levels of blood serum markers, baseline dosing formulations, inter-relationships between ingredients, and/or genotype associations. In some examples, one or more artificial intelligence models may be configured to process information from different data subsets within the one or more data sets. In examples, artificial intelligence models may be of a variety of types, for example, linear regression models, logistic regression models, polynomial regression models, linear discriminant analysis models, decision tree models, naïve bayes models, K-nearest neighbors models, learning vector quantization models, support vector machines, bagging and random forest models, and deep neural networks, for example, a sequence to sequence (seq2seq) deep neural network model (also known as a neural machine translation).

In some embodiments, artificial intelligence models may aim to learn a function that provides the most precise correlation between input values (X) and output values (Y) provided by Equation (7):

$$Y = f ( X ) \qquad ( 7 )$$

In some embodiments, artificial intelligence models may be trained using historical data sets of inputs (X) and outputs (Y) that are known to be correlated. For example, a linear regression artificial intelligence model is represented by Equation (8) provided below.

$$Y = {B}_{0} + {B}_{1} X \qquad ( 8 )$$

In some embodiments, a set of n historical data sets (Xᵢ, Yᵢ) where i=1 to n may be used to estimate the values for B₀ and B₁, as given by Equations (9) and (10).

$${B}_{1} = \frac{{Σ}_{i = 1}^{n} ( ( {X}_{i} - {\underline{X}}_{i} ) \times ( {Y}_{i} - {\underline{Y}}_{i} ) )}{{{Σ}_{i = 1}^{n} ( {X}_{i} - {\underline{X}}_{i} )}^{2}} \qquad ( 9 )$$

$${B}_{0} = \underline{{Y}_{i}} - {B}_{1} ( \underline{{X}_{i}} ) \qquad ( 10 )$$

In some embodiments, parameters B₀ and B₁, may be considered coefficients of artificial intelligence model 500. Artificial intelligence model 500 with these initial coefficients may then be used to predict the output of artificial intelligence model 500 for Y_{i,M}, given the set of historical inputs Xᵢ. Thus, Y_{i,M}, corresponds to a derived output of the artificial intelligence model 500 given Xᵢ, and may differ from a known (or “correct”) output for input Xᵢ. The error of these predictions may be calculated using Root Mean Square Error (RMSE), for example, given by Equation (11):

$$R M S E = \sqrt{{\sum }_{i = 1}^{n} \frac{{( {Y}_{i , M} - {Y}_{i} )}^{2}}{n}} \qquad ( 11 )$$

In some embodiments, training of artificial intelligence model 500 involves adjustment of the coefficients B₀ and B₁ to minimize the RMSE over multiple historical data sets (Xᵢ, Yᵢ). In some embodiments, different techniques may be used by different types of artificial intelligence models to adjust the weights (or values) of various coefficients, in general by using historical data sets that are correlated in the way that artificial intelligence model 500 is trying to predict in new data sets by minimizing the predicted error of artificial intelligence model 500 when applied to the historical data sets.

In an implementation, to determine the initial parameters that impact performance goals and measured characteristics (including blood serum marker levels) to the greatest extent, multivariate analysis is utilized. Multivariate analysis is a statistical procedure for data analysis that involves analyzing more than one dependent variable simultaneously with other variables. In some embodiments, multivariate analysis may be used to train artificial intelligence model 500 to determine previously unknown relationships between two or more pieces of biometric information, one or more pieces of biometric information together with one or more SNP, two or more SNP, one or more nutritional supplements and one or more pieces of biometric information, and one or more nutritional supplements and one or more SNP. In examples, observations over a large number of initial parameters, performance goals, and measured characteristics are collected over multiple users and over time in order to perform the multivariate analysis.

According to an implementation, gamification may be implemented through gamification engine. In an implementation, gamification may incorporate social engineering with the gamification mechanisms to incentivize the user to utilize the gamification mechanisms. According to an implementation, gamification may promote user engagement. In examples, to encourage and reward program compliance and to recognize progression towards performance goals, gamification may incentivize the user based on comparison with peer groups. In some embodiments, gamification engine 230 may employ various techniques such as leaderboards, gamifications, badges, and achievement levels to incentivize the user.

In examples, users may opt to share some aspects of their actual performance and performance goals for the purposes of competition or the creation of a leaderboard. Users may create a “handle” for anonymization while still enabling user comparisons. In examples, only the handle name of a user may be shown in the leaderboard. In examples, some additional user information may be shared, for example age or age range, gender, and general location (country, city). In examples, users may create an avatar.

In some examples, users may opt to share aspects of their physical performance to enable nutritional supplement platform 202 to form aggregate statistics which may be published or shared, for example to provide group motivation or for example to use as or to contribute to a benchmark for users to compare their individual performance to. In some examples, teams of users may be formed, and collective achievements of teams may be compared on a leaderboard. In examples, the total achievements of a group of users in a team may be aggregated and displayed. For example, on a leaderboard for weight loss, in one part of the display, it may be displayed that the total amount of weight loss of all users in the group of users of the team is 200 kg. In examples, the average achievement may be determined and displayed. For example, on a leaderboard for weight loss, in one part of the display, it may be indicated that the users in the group of users of the team have on average have lost 10 kg. In examples, the average achievement may be expressed in terms of progress towards a goal. For example, it may be indicated that on an average the team of users are 15% towards their goal. In examples, the aggregate progress towards a goal may be calculated and displayed. For example, the total amount of weight to be lost may be calculated for all users of the team and the actual amount of weight lost to date for all users of the team may be calculated. In examples the progress towards goals for all users may be calculated from these two values. In an implementation, gamification engine 230 may create a display that indicates that collectively the users (either individually or in aggregate on the team) are 30% towards their goal. This can be used to incentivize teamwork and individuals encouraging each other. In examples, teams may be formed and the collective achievements of the teams may be compared on a leaderboard. In this example, as the team achievement is most relevant, this can promote encouragement amongst members of teams. An example of achievements that may be displayed include improvements achieved (enabling absolute comparisons, for example, a total amount of weight lost (kg), finishing time of a race, personal best records, best VO2 max, etc.) In some examples, progress towards a goal may be shared for comparisons amongst individuals in a group. For example, user A has a goal to lose 30 kg, and has so far lost 6 kg. The progress of the user A towards his or her goal is 20%.

In some embodiments, personal best improvements may be compared amongst users. In an example, this may be listed as a “percentage improvement” enabling athletes from different sports to compete against each other. In examples, percentage improvements for different sports may be normalized to consider a profile of gains expected for that sport. For example, a range from low performance to high performance for a sport may be defined such that incremental improvements can be shared as a percentage of that range. In an example, for a 10 km road race, a performance range may be defined that covers 45 minutes, for example highest performance may be defined as finishing in 35 minutes, and entry level performance may be defined as finishing in 80 minutes. If an athlete's finishing time improves by 5 minutes, then the athlete may be said to have improved by 5/45×100=11%. In examples, for a high jumper, a performance range may be defined that covers 1 meter, for example highest performance may be defined as jumping 2 meters, and entry level performance may be defined as jumping 1 meter. If a second athlete's performance improves by 0.2 meter, then the athlete may be said to have improved by 0.2/1×100=20%. These two athletes could then be compared on a leaderboard for improvement.

In an implementation, gamification engine 230 may define achievement levels and award badges to users as they achieve each achievement level. In examples, achievement levels may be defined in terms of progress towards a goal or specific performance levels such as time or distance, weight lifted or other event related or general performance measures or metrics. In examples, achievement levels may be defined for 25% of goal achieved, 50% of goal achieved, 75% of goal achieved, 90% of goal achieved, and so on. In an example, when a user reaches the achievement level target, gamification engine 230 may assign a badge to the user. In examples, the badge may be shown along with the user's avatar, for example, on the user's personal dashboard or on leaderboards. In some embodiments, a physical or virtual badge may be provided to the user. In examples, users that have reached various achievement levels may be “featured” on a news reel or channel, for example for a period of time after the achievement level has been reached.

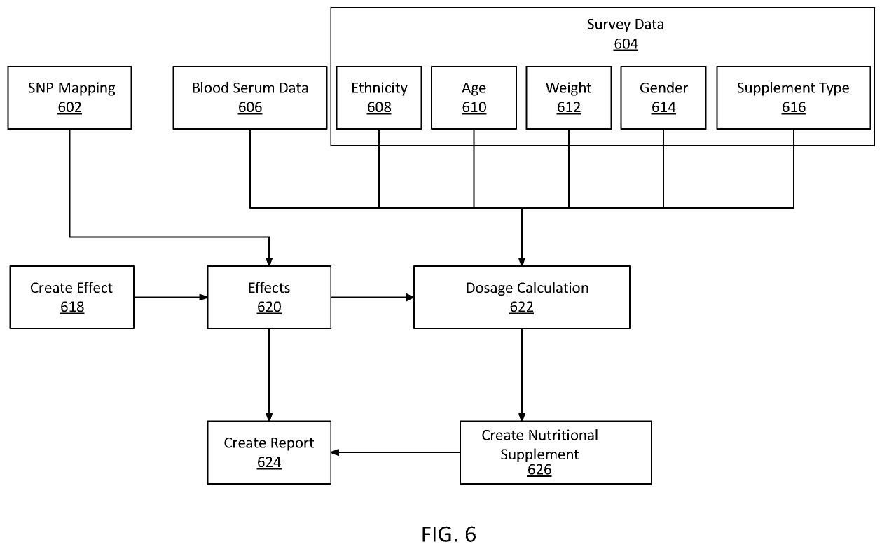

FIG. 6 depicts flow diagram 600 for creating a nutritional supplement for a user, according to one or more embodiments.

In a brief overview of an implementation of flow diagram 600, at step 602, a single nucleotide polymorphisms (SNP) mapping of the user is obtained from genomic data of the user. For example, a genetic mapping of the user may be created and/or obtained from analysis and sequencing of genetic material. In example, the genetic mapping data provided to nutritional supplement platform 202 for inclusion in the decision-making model. At step 604, survey data including health information and user preferences determined by a survey is received by nutritional supplement platform 202. In examples, users will provide information to nutritional supplement platform 202 in the form of a survey which includes questions about specific health information and user preferences, and the information may be included in the decision-making model of the nutritional supplement platform 202. In an example, the survey data includes blood serum data 606, ethnicity 608, age 610, weight 612, gender 614, and supplement type 616. In examples, the survey data may allow subjective and objective changes in the user to be measured in a quantitative and/or qualitative manner. At step 618, nutritional supplement platform 202 allows for the creation of novel ‘effects’ which are defined by specific traits or predispositions, outlined, or indicated by novel scientific research. For example, effects can be included in the model for analysis and outputs provided to the user. At step 620, data and outputs from the SNP Mapping 602 are analysed and pushed through the algorithm that separates specific SNPs from the larger genetic sequencing dataset. Additional effects that are created in step 618 following initial sampling and analysis are also queried to user mapping data 620. dosage of a nutritional supplement for the user is calculated based on the survey data 604 and in some implementations, based on the SNP Mapping 620. At step 624 a report is created for the user based on the effects. In an example, at step 626, the nutritional supplement is created for the user based on the calculated dosage. In examples, the report may be a digital insights report. In an example, the report may be a health report, a nutrition report, or both. In an example, the report may include information about the formulation of the created nutritional supplement.

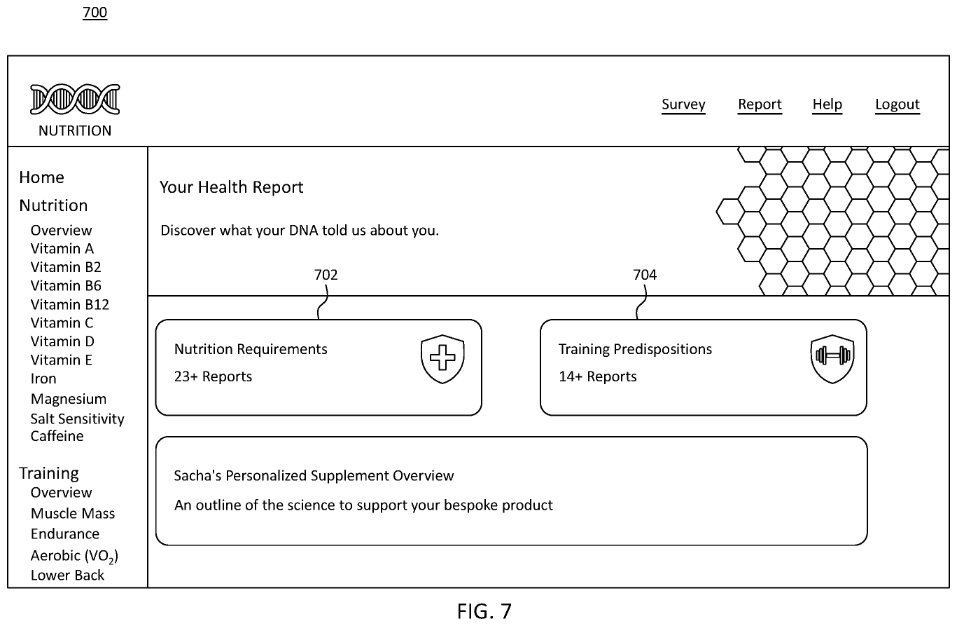

FIG. 7 depicts example 700 of a user interface that a user may use to view his or her health report and to view the profile of their personalised supplement product formulation, according to one or more embodiments. In an example, the user interface may be user interface 260-1. In examples, the health report may be generated by nutritional supplement platform 202. As shown in FIG. 7, the user is able to view nutritional requirements 702 which refers to information obtained by the SNP mapping 602 and effects 620 and in some embodiment, information pertaining to blood serum data 606. Additionally, the user will be able to view their training predispositions 704 which refers to information pertaining to their “predispositions” to response to certain stimuli (as identified by the user's SNP mapping 602 and effects 620) and may include physiological responses and rate of adaptation to exercise modalities-such as strength, endurance, power & speed-based training. In an embodiment, the example shown in FIG. 7, nutritional requirements 702 further includes more than 23 reports and training predispositions 704 includes more than 14 reports. In other embodiments, nutritional supplement platform 202 may include more than 23 reports for nutritional requirements 702 and more than 14 reports in training predispositions. In examples, the user may click on nutritional requirements 702 or training predispositions 704, for example using a mouse pointer, to view the reports.

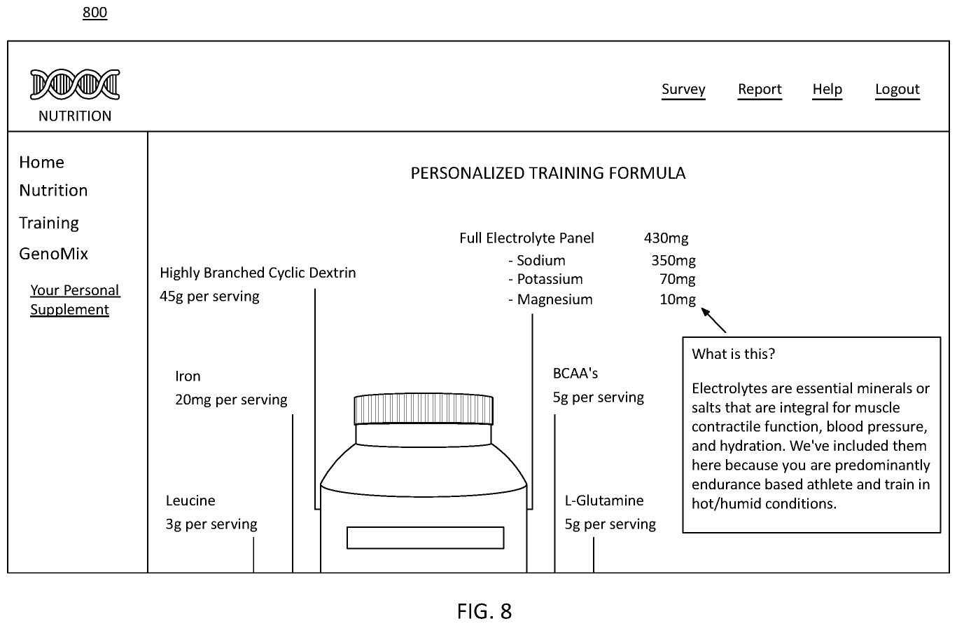

FIG. 8 depicts example 800 of a user interface that a user may use to view details of a personalized formulation of a nutritional supplement, according to one or more embodiments. In an example, the user interface may be user interface 260-1. In examples, personalized formulation of the nutritional supplement may be generated by nutritional supplement platform 202 for the user. As shown in FIG. 8, the nutritional supplement includes a plurality of ingredients. For example, the nutritional supplement includes 45 grams per serving of highly branched cyclic dextrin, 20 milligram per serving of iron, 3 gram per serving of leucine, 430 milligram of full electrolyte panel, 5 gram per serving of branched chain amino acids (BCAAs) and 3 gram per serving of Leucine, 5 gram per serving of L-Glutamine.

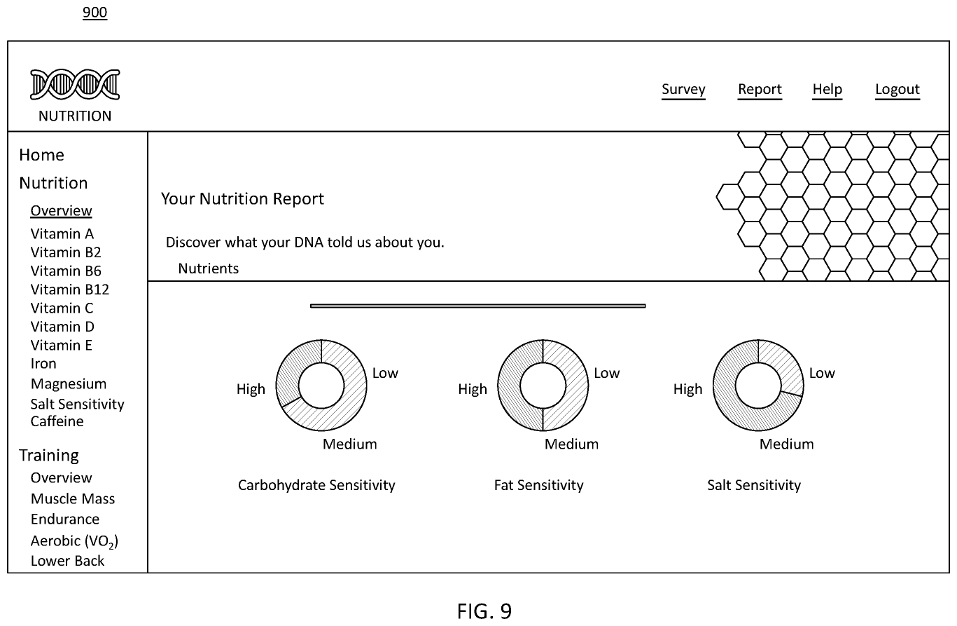

FIG. 9 depicts example 900 of a user interface that a user may use to view his or her nutrition report in a summary fashion, according to one or more embodiments. In an example, the user interface may be user interface 260-1. In examples, the nutrition report may be generated by nutritional supplement platform 202. As shown in FIG. 9, the nutrition report depicts a summary page for the nutritional requirements section 702 of the digital insights report 290 and outlines an extent of carbohydrate sensitivity, fat sensitivity, and salt sensitivity of the user as defined by a scoring mechanism defined by the SNP mapping 602. In examples, the extent could be high, medium, or low, as defined by predetermined score thresholds.

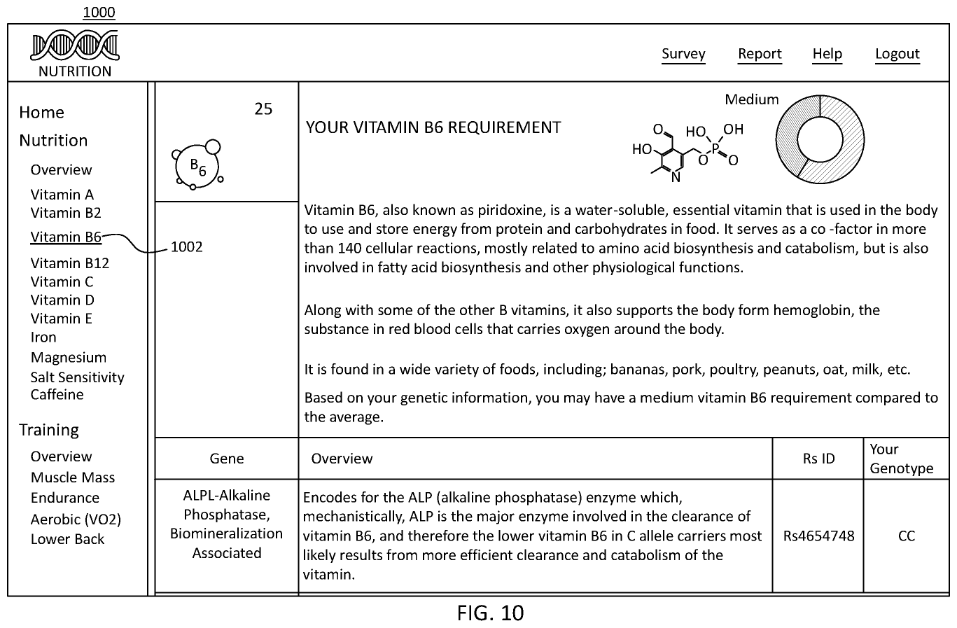

FIG. 10 depicts example 1000 of a user interface within the nutrition requirements 702 section of the digital insights report 209 and is showing vitamin B6 requirements for a user, according to one or more embodiments. As shown in FIG. 10, the nutritional supplement platform 202 shows information about vitamin B6 requirement for the user upon the user clicking on the “Vitamin B6” selectable option 1002. In examples, the report may include information to the user regarding the function of the product (in this example, vitamin B6), information regarding the user's propensity for adequate absorption and metabolism as defined by the SNP mapping 602 and in some embodiments by blood serum data 606. A score will be calculated based on the results of the SNP mapping 602 and the effects 620. In examples, the page will include a table that outlines each of the SNPs tested for, an explanation of their function and role, and the individual user's results, as defined by the SNP mapping 602.

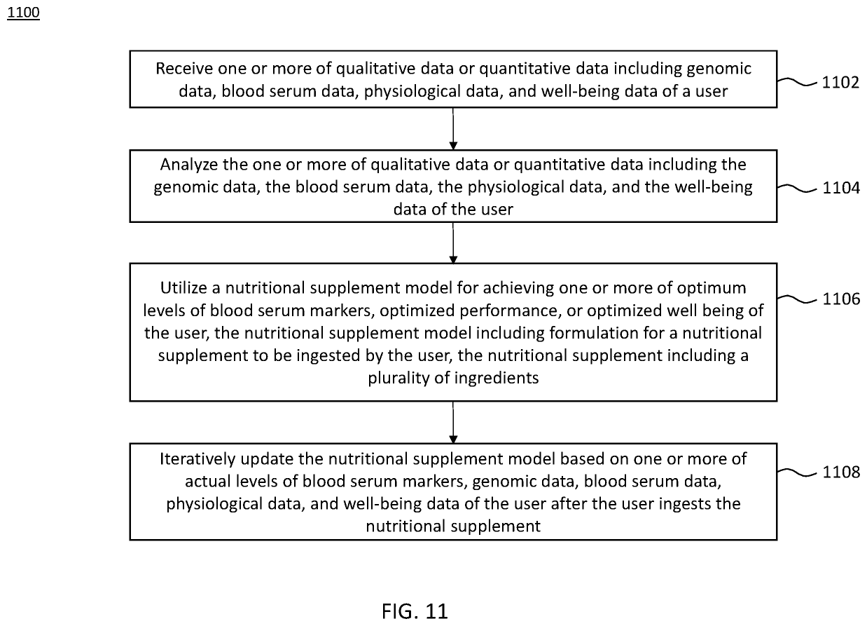

FIG. 11 depicts flowchart 1100 for iteratively updating a nutritional supplement model for a user after the user ingests a nutritional supplement, according to one or more embodiments.

In a brief overview of an implementation of flowchart 1100, at step 1102, one or more of qualitative data or quantitative data including genomic data, blood serum data, physiological data, and well-being data of a user may be received. At step 1104, the one or more of qualitative data or quantitative data including the genomic data, the blood serum data, the physiological data, and the well-being data of the user may be analyzed. At step 1106, a nutritional supplement model for achieving one or more of optimum levels of blood serum markers, optimized performance, or optimized well-being of the user may be utilized, the nutritional supplement model including formulation for a nutritional supplement to be ingested by the user, the nutritional supplement including a plurality of ingredients. At step 1108, the nutritional supplement model may be iteratively updated based on one or more of actual levels of blood serum markers, genomic data (original or updated), blood serum data, physiological data, and well-being data of the user after the user ingests the nutritional supplement.

Step 1102 includes receiving one or more of qualitative data or quantitative data including genomic data, blood serum data, physiological data, and well-being data of a user. According to an implementation, data analyzer 224 may be configured to receive one or more of qualitative data or quantitative data including genomic data, blood serum data, physiological data, and well-being data of the user. In examples, the genomic data may be generated based on genetic material of the user. According to an implementation, a genetic material sampler 220 may be configured to generate the genomic data based on genetic material of the user. Further, in examples, blood serum analyzer 222 may be configured to generate the blood serum data of the user.

Step 1104 includes analyzing the one or more of qualitative data or quantitative data including the genomic data, the blood serum data, the physiological data, and the well-being data of the user. According to an implementation, data analyzer 224 may be configured to analyze the one or more of qualitative data including the genomic data, the blood serum data, the physiological data, and the well-being data of the user.

Step 1106 includes utilizing a nutritional supplement model for one or more of achieving optimum levels of blood serum markers, optimizing performance, or optimized well-being of the user, the nutritional supplement model including formulation for a nutritional supplement to be ingested by the user, the nutritional supplement including a plurality of ingredients. According to an implementation, data analyzer 224 may be configured to utilize the nutritional supplement model for achieving optimum levels of blood serum markers. According to an implementation, data analyzer 224 may be configured to utilize the nutritional supplement model for achieving optimized performance of the user, for example for enabling a user to achieved one or more performance goals. According to an implementation, data analyzer 224 may be configured to utilize the nutritional supplement model for achieving optimized well-being of the user, for example based on one or more responses of the user to a user survey. In an example, step 1106 includes utilizing a nutritional supplement model for achieving optimum dosages of nutritional supplement ingredients through alignment with of one or more of the following components, blood serum markers optimization, physical performance measurements, and subjective quantitative and/or qualitative survey feedback. In examples, the nutritional supplement model including formulation for a nutritional supplement to be ingested by the user, the nutritional supplement including a plurality of ingredients.

Step 1108 includes iteratively updating the nutritional supplement model based on one or more of actual levels of blood serum markers, genomic data, blood serum data, physiological data, and well-being data of the user after the user ingests the nutritional supplement. According to an implementation, model updation engine 228 may be configured to iteratively update the nutritional supplement model based on one or more of actual levels of blood serum markers, genomic data, blood serum data, physiological data, and well-being data of the user after the user ingests the nutritional supplement. In examples, step 1108 includes iteratively updating the nutritional supplement model based on one or more of the following components-actual levels of blood serum markers, genomic data, blood serum data, physiological data, and well-being data of the user after the user ingests the nutritional supplement.

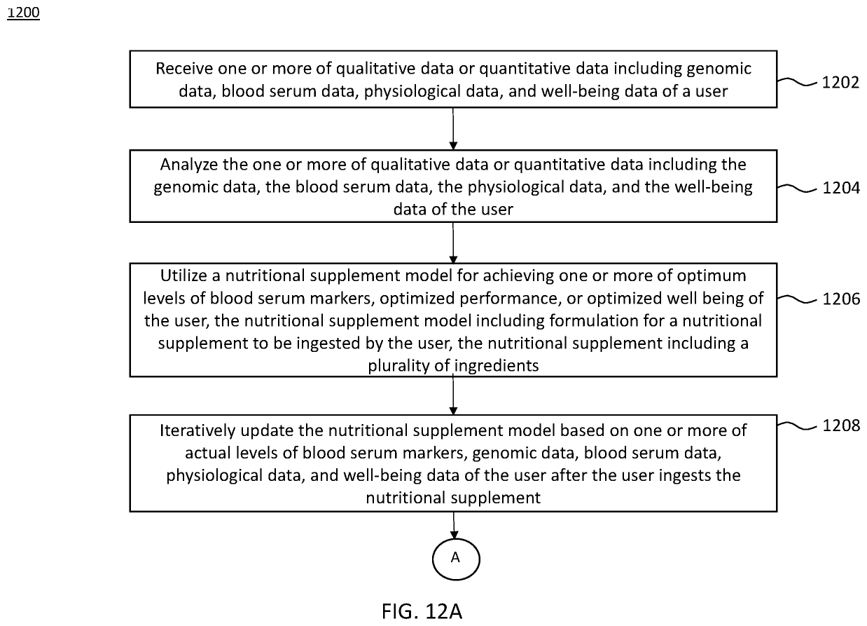

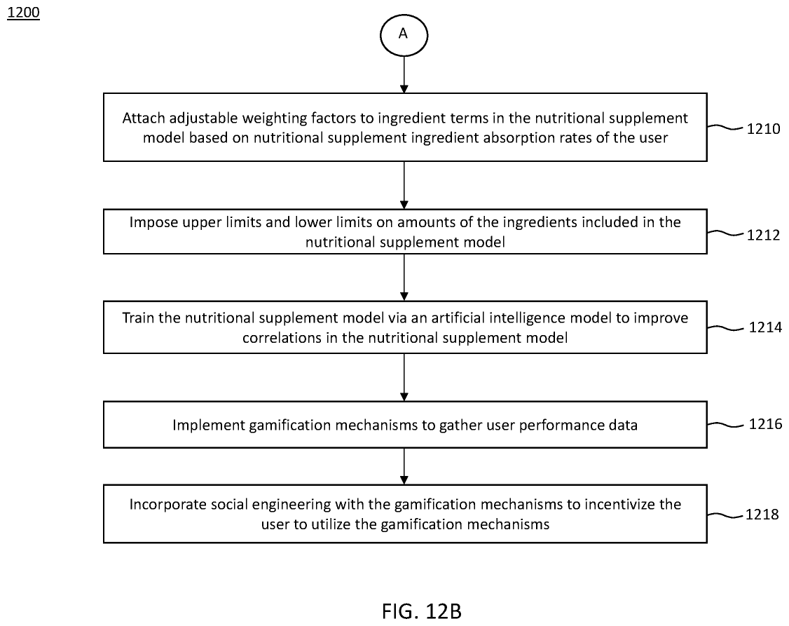

FIG. 12A and FIG. 12B depict flowchart 1200 for training a nutritional supplement model via an artificial intelligence model to improve correlations in the nutritional supplement model, according to one or more embodiments.

In a brief overview of an implementation of flowchart 1200, at step 1202, one or more of qualitative data or quantitative data including genomic data, blood serum data, physiological data, and well-being data of a user may be received. At step 1204, the one or more of qualitative data or quantitative data including the genomic data, the blood serum data, the physiological data, and the well-being data of the user may be analyzed. At step 1206, a nutritional supplement model for achieving one or more of optimum levels of blood serum markers, optimized performance of the user, or optimized well-being of the user may be utilized, the nutritional supplement model including formulation for a nutritional supplement to be ingested by the user, the nutritional supplement including a plurality of ingredients. At step 1208, the nutritional supplement model may be iteratively updated based on one or more of actual levels of blood serum markers, genomic data, blood serum data, physiological data, and well-being data of the user after the user ingests the nutritional supplement. At step 1210, adjustable weighting factors may be attached to ingredient terms in the nutritional supplement model based on nutritional supplement ingredient absorption rates of the user. At step 1212, upper limits and lower limits may be imposed on amounts of the ingredients included in the nutritional supplement model. At step 1214, the nutritional supplement model may be trained via an artificial intelligence model to improve correlations in the nutritional supplement model. At step 1216, user performance data may be gathered via gamification mechanisms. At step 1218, the user may be incentivized to utilize the gamification mechanisms via social engineering.

Step 1202 includes receiving one or more of qualitative data or quantitative data including genomic data, blood serum data, physiological data, and well-being data of a user. According to an implementation, data analyzer 224 may be configured to receive one or more of qualitative data or quantitative data including genomic data, blood serum data, physiological data, and well-being data of the user. In examples, the genomic data may be generated based on genetic material of the user. According to an implementation, a genetic material sampler 220 may be configured to generate the genomic data based on genetic material of the user. Further, in examples, blood serum analyzer 222 may be configured to generate the blood serum data of the user.

Step 1204 includes analyzing the one or more of qualitative data or quantitative data including the genomic data, the blood serum data, the physiological data, and the well-being data of the user. According to an implementation, data analyzer 224 may be configured to analyze the one or more of qualitative data including the genomic data, the blood serum data, the physiological data, and the well-being data of the user.

Step 1206 includes utilizing a nutritional supplement model for achieving one or more of optimum levels of blood serum markers, optimized performance levels, or optimized well-being of the user, the nutritional supplement model including formulation for a nutritional supplement to be ingested by the user, the nutritional supplement including a plurality of ingredients. According to an implementation, data analyzer 224 may be configured to utilize the nutritional supplement model for achieving optimum levels of blood serum markers. According to an implementation, data analyzer 224 may be configured to utilize the nutritional supplement model for achieving optimized performance of the user, for example as determined based on the user achieving one or more performance goals. According to an implementation, data analyzer 224 may be configured to utilize the nutritional supplement model for achieving optimized well-being of the user, for example as determined based on user responses to a user survey or by assessing quantitative and/or qualitative user survey feedback. In an example, step 1206 includes utilizing a nutritional supplement model for achieving optimum dosages of nutritional supplement ingredients through alignment with of one or more of the following components, blood serum markers optimization, physical performance measurements, and subjective quantitative and/or qualitative survey feedback. In examples, the nutritional supplement model including formulation for a nutritional supplement to be ingested by the user, the nutritional supplement including a plurality of ingredients.

Step 1208 includes iteratively updating the nutritional supplement model based on one or more of actual levels of blood serum markers, genomic data, blood serum data, physiological data, and well-being data of the user after the user ingests the nutritional supplement. According to an implementation, model updation engine 228 may be configured to iteratively update the nutritional supplement model based on one or more actual levels of blood serum markers, genomic data (existing or updated), blood serum data, physiological data, and well-being data of the user after the user ingests the nutritional supplement. In examples, step 1208 includes iteratively updating the nutritional supplement model based on one or more of the following components-actual levels of blood serum markers, genomic data, blood serum data, physiological data, and well-being data of the user after the user ingests the nutritional supplement.

Step 1210 includes attaching adjustable weighting factors to ingredient terms in the nutritional supplement model based on nutritional supplement ingredient absorption rates of the user. According to an implementation, data analyzer 224 may be configured to attach adjustable weighting factors to ingredient terms in the nutritional supplement model based on nutritional supplement ingredient absorption rates of the user.

Step 1212 includes imposing upper limits and lower limits on amounts of the ingredients included in the nutritional supplement model. According to an implementation, data analyzer 224 may be configured to impose upper limits and lower limits on amounts of the ingredients included in the nutritional supplement model.

Step 1214 includes training the nutritional supplement model via an artificial intelligence model to improve correlations in the nutritional supplement model. According to an implementation, model training engine 226 may be configured to train the nutritional supplement model via the artificial intelligence model to improve correlations in the nutritional supplement model. In examples, the artificial intelligence model may be a machine learning model. Further, in examples, training the nutritional supplement model includes incorporating data sets of a population. In an example, the artificial intelligence model may be selected from the group consisting of a linear regression model, a logistic regression model, a polynomial regression model, a linear discriminant analysis model, a decision tree model, a naïve bayes model, a K-nearest neighbor model, a learning vector quantization model, a support vector machine, a bagging and random forest model, and a deep neural network.

Step 1216 includes gathering user performance data via gamification mechanisms. According to an implementation, gamification engine 230 may be configured to implement gamification mechanisms to gather user performance data.

Step 1218 includes incentivizing the user to utilize the gamification mechanisms via social engineering. According to an implementation, gamification engine 230 may be configured to incorporate social engineering with the gamification mechanisms to incentivize the user to utilize the gamification mechanisms.

In examples, systems and methods described above may provide multiple ones of any or each of those components and these components may be provided on either a standalone machine or, in some embodiments, on multiple machines in a distributed system. The systems and methods described above may be implemented as a method, apparatus or article of manufacture using programming and/or engineering techniques to produce software, firmware, hardware, or any combination thereof. In addition, the systems and methods described above may be provided as one or more computer-readable programs embodied on or in one or more articles of manufacture. The term “article of manufacture” as used herein is intended to encompass code or logic accessible from and embedded in one or more computer-readable devices, firmware, programmable logic, memory devices (e.g., EEPROMs, ROMs, PROMS, RAMS, SRAMs, etc.), hardware (e.g., integrated circuit chip, Field Programmable Gate Array (FPGA), Application Specific Integrated Circuit (ASIC), etc.), electronic devices, a computer readable non-volatile storage unit (e.g., CD-ROM, floppy disk, hard disk drive, etc.). The article of manufacture may be accessible from a file server providing access to the computer-readable programs via a network transmission line, wireless transmission media, signals propagating through space, radio waves, infrared signals, etc. The article of manufacture may be a flash memory card or a magnetic tape. The article of manufacture includes hardware logic as well as software or programmable code embedded in a computer readable medium that is executed by a processor. In general, computer-readable programs may be implemented in any programming language, such as LISP, PERL, C, C++, C#, PROLOG, or in any byte code language such as JAVA. The software programs may be stored on or in one or more articles of manufacture as object code.

While various embodiments of the methods and systems have been described, these embodiments are illustrative and in no way limit the scope of the described methods or systems. Those having skill in the relevant art can effect changes to form and details of the described methods and systems without departing from the broadest scope of the described methods and systems. Thus, the scope of the methods and systems described herein should not be limited by any of the illustrative embodiments and should be defined in accordance with the accompanying claims and their equivalents.

What is claimed is:

1. A system for implementing a nutritional supplement plan for a user, the system comprising:
a non-transitory memory configured to store one or more of qualitative data or quantitative data including genomic data, blood serum data, physiological data, and well-being data of the user; and
a processor configured to:
analyze the qualitative data or quantitative data including genomic data, blood serum data, physiological data, and well-being data of the user;
utilize a nutritional supplement model for achieving optimized performance or well-being of the user, the nutritional supplement model including formulation for a nutritional supplement to be ingested by the user, the nutritional supplement including a plurality of ingredients; and
iteratively update the nutritional supplement model based on one or more of actual levels of blood serum markers, genomic data, blood serum data, physiological data, and well-being data of the user after the user ingests the nutritional supplement.

2. The system of claim 1, wherein the processor is further configured to train the nutritional supplement model via an artificial intelligence model to improve correlations in the nutritional supplement model.

3. The system of claim 2, wherein the artificial intelligence model is a machine learning model incorporating data sets of a population.

4. The system of claim 2, wherein the artificial intelligence model is selected from the group consisting of a linear regression model, a logistic regression model, a polynomial regression model, a linear discriminant analysis model, a decision tree model, a naïve bayes model, a K-nearest neighbor model, a learning vector quantization model, a support vector machine, a bagging and random forest model, and a deep neural network, and combinations thereof.

5. The system of claim 1, wherein the processor is further configured to implement gamification mechanisms to gather user performance data.

6. The system of claim 5, wherein the gamification mechanisms incorporate social engineering with the gamification mechanisms to incentivize the user to utilize the gamification mechanisms.

7. The system of claim 1, wherein the processor is further configured to attach adjustable weighting factors to ingredient terms in the nutritional supplement model based on nutritional supplement ingredient absorption rates of the user.

8. The system of claim 7, wherein the processor is further configured to impose upper limits and lower limits on amounts of the ingredients included in the nutritional supplement model.

9. The system of claim 1, further comprising a genetic material sampler configured to generate the genomic data based on genetic material of the user.

10. The system of claim 1, further comprising a blood serum analyzer configured to generate the blood serum data of the user.

11. A method for implementing a nutritional supplement plan for a user, the method comprising steps of:
receiving one or more of qualitative data or quantitative data including genomic data, blood serum data, physiological data, and well-being data of the user;
analyzing the qualitative data or quantitative data including genomic data, blood serum data, physiological data, and well-being data of the user;
utilizing a nutritional supplement model for achieving optimized performance or well-being of the user, the nutritional supplement model including formulation for a nutritional supplement to be ingested by the user, the nutritional supplement including a plurality of ingredients; and
iteratively updating the nutritional supplement model based on one or more of actual levels of blood serum markers, genomic data, blood serum data, physiological data, and well-being data of the user after the user ingests the nutritional supplement.

12. The method of claim 11, further comprising a step of training the nutritional supplement model via an artificial intelligence model to improve correlations in the nutritional supplement model.

13. The method of claim 12, wherein the artificial intelligence model is a machine learning model and the step of training the nutritional supplement model includes a step of incorporating data sets of a population.

14. The method of claim 12, wherein the artificial intelligence model is selected from the group consisting of a linear regression model, a logistic regression model, a polynomial regression model, a linear discriminant analysis model, a decision tree model, a naïve bayes model, a K-nearest neighbor model, a learning vector quantization model, a support vector machine, a bagging and random forest model, and a deep neural network or combinations thereof.

15. The method of claim 11, further comprising a step of gathering user performance data via gamification mechanisms.

16. The method of claim 15, wherein the user performance data gathering step includes a step of incentivizing the user to utilize the gamification mechanisms via social engineering.

17. The method of claim 11, further comprising a step of attaching adjustable weighting factors to ingredient terms in the nutritional supplement model based on nutritional supplement ingredient absorption rates of the user.

18. The method of claim 17, further comprising a step of imposing upper limits and lower limits on amounts of the ingredients included in the nutritional supplement model.

19. The method of claim 11, further comprising a step of generating the genomic data based on genetic material of the user via a genetic material sampler.

20. The method of claim 11, further comprising a step of generating the blood serum data of the user via a blood serum analyzer.

21. A system for implementing a nutritional supplement plan for a user, the system comprising:
a genetic material sampler configured to generate genomic data based on genetic material of the user;
a blood serum analyzer configured to generate blood serum data of the user;
a non-transitory memory configured to store qualitative data including the genomic data, the blood serum data, physiological data, and well-being data of the user; and
a processor configured to:
analyze the qualitative data including genomic data, blood serum data, physiological data, and well-being data of the user;
construct a nutritional supplement model for achieving optimized performance or well-being of the user, the nutritional supplement model including formulation for a nutritional supplement to be ingested by the user, the nutritional supplement including a plurality of ingredients;
iteratively update the nutritional supplement model based on one or more of actual levels of blood serum markers, genomic data, blood serum data, physiological data, and well-being data of the user after the user ingests the nutritional supplement;
attach adjustable weighting factors to ingredient terms in the nutritional supplement model based on nutritional supplement ingredient absorption rates of the user;
impose upper limits and lower limits on amounts of the ingredients included in the nutritional supplement model;
train the nutritional supplement model via an artificial intelligence model to improve correlations in the nutritional supplement model;
implement gamification mechanisms to gather user performance data; and
incorporate social engineering with the gamification mechanisms to incentivize the user to utilize the gamification mechanisms.
